(19) United States
(12) Patent Application Publication
(10) Pub. No.: US 2023/0288558 A1
(43) Pub. Date: Sep. 14, 2023

(54) SYSTEMS AND METHODS FOR SINGLE TRANSDUCER HARMONIC MOTION IMAGING

(71) Applicant: The Trustees of Columbia University in the City of New York, New York, NY (US)

(72) Inventors: Elisa E. KONOFAGOU, New York, NY (US); Md Murad Hossain, New York, NY (US)

(73) Assignee: The Trustees of Columbia University in the City of New York, New York, NY (US)

(21) Appl. No.: 18/064,021

(22) Filed: Dec. 9, 2022

Related U.S. Application Data
(63) Continuation of application No. PCT/US2021/037639, filed on Jun. 16, 2021.
(60) Provisional application No. 63/120,469, filed on Dec. 2, 2020.
(60) Provisional application No. 63/039,839, filed on Jun. 16, 2020.

(51) Int. Cl. G01S 15/89; G01S 15/10; G01S 7/62; G01S 7/52; A61B 8/08

(52) U.S. Cl. CPC G01S 15/8911; G01S 15/102; G01S 7/629; G01S 7/52095; G01S 7/52036; A61B 8/08

(57) ABSTRACT

The present subject matter relates to techniques for single transducer harmonic motion imaging. The disclosed system can include a transducer. The transducer can be configured to generate an amplitude-modulated acoustic radiation force (AM-ARF) by sinusoidally modulating a duration of an excitation pulse of an acoustic radiation force, induce a harmonic motion on a target tissue using the AM-ARF, and simultaneously track the harmonic motion by collecting a tracking pulse. The tracking pulse can be interleaved between the excitation pulse.

### CROSS-REFERENCE TO RELATED APPLICATION

This application is a continuation of International Patent Application No. PCT/US2021/037639 filed Jun. 16, 2021, which claims priority to U.S. Provisional Patent Application Nos. 63/039,839, which was filed on Jun. 16, 2020, and 63/120,469, which was filed on Dec. 2, 2020, the entire contents of which are incorporated by reference herein.

### GRANT INFORMATION

This invention was made with government support under grant number 5R01CA228275 from the National Institutes of Health (NIH). The government has certain rights in the invention

### BACKGROUND

Ultrasound elastography (UE), magnetic resonance elastography (MRE), or optical coherence elastography (OCE) derived mechanical properties can be used to diagnose diseases, monitor the efficacy of treatment, and plan the surgery. These elastographic methods are different in terms of the use of the mechanical force to probe the tissue, tracking force-induced deformation, and inferring mechanical properties from the estimated deformation. Due to these differences, the estimated mechanical properties and the perceived size of the lesions/inclusions vary between different elastographic methods. While these variations can be mitigated by assessing mechanical properties as a function of frequency, interrogated frequencies are also different among these methods. As an example, MRE uses the single frequency shear wave (i.e., harmonic shear waves) in the 20-60 Hz range, whereas generated shear waves in the ultrasound elastography can be harmonic or transient/impulsive (i.e., broadband frequency range of 50-2000 Hz).

The UE can be used in various applications due to its low cost, ease of use, portability, real-time capability, ability to penetrate deeper in tissue, and ability to characterize the motion within the human body. Acoustic radiation force (ARF) can be used to induce motion within the tissue during certain UE applications. Certain ARF-based methods can use displacements “on-axis” to the ARF, shear wave propagation “off-axis”to the ARF, or both to assess the mechanical properties. However, both “on-axis” and “off-axis”-based methods have certain limitations. For example, shear wave-based methods provide quantitative mechanical properties like elasticity and viscosity. However, the shear wave based methods suffer from tissue heterogeneities and reflected shear waves, poor mechanical resolution, and less penetration depth. Shear wave velocity as a function of frequency (i.e., phase velocity) was used to phase velocity dispersion due to viscosity and ARF geometry. The selection of frequencies is important to correctly estimate mechanical properties and detect inclusion. Although ARF “on-axis” displacements, similar to shear wave velocity, can depend on the frequency, currently available techniques can be applied at the limited frequency ranges.

Therefore, there is a need for improved ARF elastography techniques to interrogate mechanical properties of tissue either using “on-axis” displacements or “off-axis” shear wave velocity at a wider frequency range.

### SUMMARY

The disclosed subject matter provides techniques for harmonic motion imaging. The disclosed subject matter provides systems and methods for single transducer harmonic motion imaging.

In certain embodiments, an example system for single transducer harmonic motion imaging can include a transducer. The transducer can be configured to generate an amplitude-modulated acoustic radiation force (AM-ARF) by sinusoidally modulating a duration of an excitation pulse of an acoustic radiation force, induce a harmonic motion on a target tissue using the AM-ARF, and simultaneously track the harmonic motion by collecting a tracking pulse. In non-limiting embodiments, the system can include a processor configured to estimate the mechanical properties of the target tissue based on the tacked harmonic motion. In non-limiting embodiments, the tracking pulse can be interleaved between the excitation pulse.

In certain embodiments, the excitation pulse can include a sum of sinusoids with about 100-2000 Hz frequencies. In non-limiting embodiments, a cycle of the excitation pulse can be at least about 2 cycles.

In certain embodiments, a cycle of the tracking pulse can be about 2 cycles. In non-limiting embodiments, a pulse repetition frequency (PRF) of the tracking pulse can be from about 10 kHz to about 20 kHz.

In certain embodiments, the processor can be configured to generate a displacement map corresponding to the frequencies of the excitation pulse. In non-limiting embodiments, the processor can be configured to generate beamformed radiofrequency (RF) data by performing delay-and-sum beamforming, estimate displacements induced by the AM-ARF based on the RF data, perform a two-dimensional interpolation on the estimated displacements, calculate differential displacements between successive time points based on interpolated displacements, filter the differential displacements for target frequencies, and generate peak-to-peak displacement (P2PD) image based on the filtered differential displacements.

In certain embodiments, the target tissue can be cancer, tumor, brain tissue, liver tissue, pancreatic tissue, breast tissue, prostate tissue, heart tissue, or combinations thereof.

In certain embodiments, the mechanical properties can include stiffness, Young's modulus, elasticity, viscosity, porosity, permeability, degree of anisotropy, or combinations thereof.

In certain embodiments, the transducer can be configured to generate AM-ARF-induced displacements at a plurality of frequencies simultaneously.

In certain embodiments, the transducer can be configured to generate P2PD at a predetermined at two orthogonal directions by mechanically rotating a linear array transducer or electronically rotating a point spread function using a matrix array or a transducer with more than one element in an elevational direction or a row-column array. In non-limiting embodiments, the point spread function can define a shape of an ultrasound beam.

In certain embodiments, the degree of anisotropy can be calculated based on the P2PD at the orthogonal directions.

In certain embodiments, a plurality of P2PDs at more than one frequencies can be generated at two orthogonal directions by mechanically rotating a linear array transducer or electronically rotating point spread function using a matrix array or a transducer with more than one element in an elevational direction or a row-column array, wherein the P2PDs at the orthogonal directions can be used to derive a degree of anisotropy as a function of frequency.

In certain embodiments, the processor can be configured to derive a degree of anisotropy by fitting a P2PD versus frequency relationship derived analytically or empirically using an anisotropic material model without rotating transducer or point spread function.

In certain embodiments, the disclosed subject matter provides methods for single transducer harmonic motion imaging. An example method can include generating an amplitude-modulated acoustic radiation force (AM-ARF) at a predetermined frequency by sinusoidally modulating a duration of an excitation pulse of an acoustic radiation force, inducing a harmonic motion on a target tissue using the AM-ARF, simultaneously tracking the harmonic motion by collecting a tracking pulse, and estimating the mechanical properties of the target tissue based on the tacked harmonic motion. In non-limiting embodiments, the tracking pulse can be interleaved between the excitation pulse.

In certain embodiments, the method can further include generating beamformed radiofrequency (RF) data by performing delay-and-sum beamforming, estimating displacements induced by the AM-ARF based on the RF data, performing a two-dimensional interpolation on the estimated displacements, calculating differential displacements between successive time points based on interpolated displacements, filtering the differential displacements for target frequencies, and generating peak-to-peak displacement (P2PD) image based on the filtered differential displacements.

In certain embodiments, the target tissue can include cancer, tumor, brain tissue, liver tissue, pancreatic tissue, breast tissue, prostate tissue, heart tissue, arterial tissue, renal tissue, or combinations thereof.

In certain embodiments, the method can further include determining a metastatic location and a metastatic level based on the estimated mechanical properties and administering a treatment to the metastatic location based on the metastatic level. In non-limiting embodiments, the treatment can include anti-tumor treatment, anti-cancer treatment, chemotherapy, immunotherapy, radiation, surgery, or combinations thereof.

In certain embodiments, the method can further include generating AM-ARF-induced displacements at a plurality of frequencies simultaneously.

In certain embodiments, the method can further include generating a displacement map corresponding to the frequencies of the excitation pulse.

In certain embodiments, the excitation pulse can include a sum of sinusoids with about 100-2000 Hz frequencies. In non-limiting embodiments, a pulse repetition frequency (PRF) of the tracking pulse can be from about 10 kHz to about 20 kHz. In some embodiments, the mechanical properties include stiffness, Young's modulus, elasticity, viscosity, porosity, permeability, degree of anisotropy, or combinations thereof.

In certain embodiments, P2PD at a predetermined frequency can be generated at two orthogonal directions by mechanically rotating a linear array transducer or electronically rotating a point spread function using a fully sampled matrix array or a transducer with more than one element in an elevational direction or a row-column array.

In certain embodiments, the degree of anisotropy can be calculated based on the P2PD at the orthogonal directions.

In certain embodiments, the disclosed subject matter provides a method for generating a phase velocity map. The method can include generating an amplitude-modulated acoustic radiation force (AM-ARF) by sinusoidally modulating a duration of an excitation pulse of an acoustic radiation force, inducing shear waves at one or more frequencies simultaneously on a target tissue using the AM-ARF, tracking the shear wave by collecting a tracking plane wave frames at a plurality angles with a frame rate between about 10 to about 20 kHz or collecting a focused pulse at a pulse repetition frequency of from about 10 to about 20 kHz between the excitation pulses, generating a phase velocity map based on the tracked shear wave, and estimating mechanical properties of the target tissue based on the phase velocity map. In non-limiting embodiments, the excitation pulse can include at least about 2 cycles of the lowest harmonic frequency.

In certain embodiments, the method can further include generating beamformed radiofrequency (RF) data by performing delay-and-sum beamforming, estimating displacements induced by the AM-ARF based on the RF data, performing a two-dimensional interpolation on the estimated displacements, calculating differential displacements between successive time points based on interpolated displacements, perform filtering on the calculated differential displacements to remove reflected shear waves, performing one dimensional Fourier transformation on the filtered differential displacements along time dimension to select a frequency of the excitation pulse, performing two-dimensional Fourier transform on the along spatial dimension at the selected frequency of the excitation pulse, and calculating a phase velocity at the selected frequency based on properties of the time and spatially Fourier transformed differential displacements. In non-limiting embodiments, the properties of the time and spatially Fourier transformed differential displacements can include a wave number, a temporal frequency, a linear regression of phase versus distance. or combinations thereof.

In certain embodiments, the shear waves at one or more frequencies can be generated at two orthogonal directions by mechanically rotating a linear array transducer or electronically rotating point spread function using a matrix array or a transducer with higher number of lateral versus elevational elements or row-column array. In non-limiting embodiments, the shear waves at the orthogonal directions can be used to derive a degree of anisotropy as a function of frequency.

In certain embodiments, the method can include generating quantitative mechanical properties of the target tissue by using P2PD versus frequency or phase velocity versus frequency relationship of by using a rheological model. In non-limiting embodiments, rheological model can be the Maxwell Model, Kelvin-Voigt model, standard linear solid model, Burgers model, generalized Maxwell model or Prony series.

In certain embodiments, the method can further include determining a metastatic location and a metastatic level based on the estimated mechanical properties and administering a treatment to the metastatic location based on the metastatic level.

In certain embodiments, the mechanical properties can include stiffness, Young's modulus, elasticity, viscosity, porosity, permeability, a degree of anisotropy, or combinations thereof. In non-limiting embodiments, the treatment includes anti-tumor treatment, anti-cancer treatment, chemotherapy, immunotherapy, radiation, surgery, or combinations thereof. In non-limiting embodiments, the target tissue can be cancer, tumor, brain tissue, liver tissue, pancreatic tissue, breast tissue, prostate tissue, heart tissue, arterial tissue, or combinations thereof.

The disclosed subject matter will be further described below.

### BRIEF DESCRIPTION OF THE DRAWINGS

FIG. 1 provides a diagram of an example method in accordance with the disclosed subject matter.

FIGS. 2A-2B provide graphs (FIG. 2A) continuous excitation pulse composed of a sum of sinusoids with 100:100:1000 Hz frequencies and (FIG. 2B) pulse sequence with the duration of discrete excitation and tracking pulses in accordance with the disclosed subject matter.

FIGS. 3A-3E provide images and graphs showing (FIG. 3A) B-mode ultrasound image of 6.5 mm, 36 kPa inclusion, single transducer harmonic motion imaging (ST-HMI) derived (FIG. 3B) displacement profiles, (FIG. 3C) differential displacement between successive time points, (FIG. 3D) magnitude spectrum of fast Fourier transform (FFT) of the differential displacement profiles, (FIG. 3E) filtered displacement profiles at 300 Hz in 36 kPa inclusion and 18 kPa background in accordance with the disclosed subject matter.

FIG. 4 provides images showing B mode, acoustic radiation force impulse (ARFI)-derived normalized peak displacement, and ST-HMI derived normalized peak-to-peak displacement image at 100:100:1000 Hz of 36 kPa inclusion with 10.4 mm (1st-2^{nd} rows), 6.5 mm (3rd-4^{th} rows), 2.5 mm (5th-6^{th} rows), and 1.6 mm (7th-8^{th} rows) diameters in accordance with the disclosed subject matter.

FIGS. 5A-5D provides graphs showing the contrast of ARFI and ST-HMI derived images at 100:100:1000 Hz of 6, 9, 36, and 70 kPa inclusions with 10.4, 6.5, 2.5, and 1.6 mm diameters in accordance with the disclosed subject matter.

FIGS. 6A-6D provides graphs showing contrast to noise ratio (CNR) of ARFI, and ST-HMI derived images at 100:100:1000 Hz of 6, 9, 36, and 70 kPa inclusions with 10.4, 6.5, 2.5, and 1.6 mm diameters in accordance with the disclosed subject matter.

FIG. 7 provides a graph showing ST-HMI-derived Peak-to-peak displacement (P2PD) and ARFI-derived peak displacement (PD) ratio of background (BKD) to inclusion (INC) versus Young's moduli ratio of inclusion to the background with R² value in accordance with the disclosed subject matter.

FIG. 8 provides images showing the B-mode and normalized peak-to-peak displacement image overlaid on the B-mode ultrasound image of mouse tumor in accordance with the disclosed subject matter.

FIG. 9 provides a graph showing example ST-HMI pulse sequences with the duration of excitation and tracking pulse for 220 Hz oscillation frequency, 0.2 ms offset, and 8 excitation pulses per cycle. In accordance with the disclosed subject matter.

FIG. 10 provides a diagram showing an example method of data processing in accordance with the disclosed subject matter.

FIGS. 11A-11D provide graphs showing ST-HMI derived (FIG. 11A) displacement profiles, (FIG. 11B) differential displacement between successive time points, (FIG. 11C) magnitude spectrum of fast Fourier transform (FFT) of the differential displacement profiles, (FIG. 11D) filtered displacement profiles in 15 kPa inclusion and 5 kPa background in accordance with the disclosed subject matter.

FIGS. 12A-12C provide images showing (FIG. 12A) ST-HMI derived peak-to-peak displacement (P2PD) image of a 15 KPa inclusion embedded in 5 kPa background, (FIG. 12B) axial distance versus median P2PD over a lateral distance of [−10−8.2] and [7.9 9.7] mm, and (FIG. 12C) normalized P2PD of the same inclusion in accordance with the disclosed subject matter.

FIG. 13 provides images showing ST-HMI derived normalized peak-to-peak images of 8, 10, 14, 15, 20, 40, and 60 kPa inclusions embedded in 5 kPa background of two commercial phantoms in accordance with the disclosed subject matter.

FIGS. 14A-14B provide graphs showing (FIG. 14A) contrast and CNR versus Young's moduli ratio of inclusion over background and (FIG. 14B) peak-to-peak displacement ratio of background over inclusion versus Young's moduli ratio of inclusion over background over R² value in accordance with the disclosed subject matter.

FIGS. 15A-15F provide graphs showing contrast and CNR of ST-HMI derived peak-to-peak displacement images of 15 kPA and 60 kPa inclusions versus (FIGS. 15A and 15D) HMI excitation duty cycle, (FIGS. 15B and 15E) oscillation cycle number, and (FIGS. 15C and 15F) excitation pulse offset in accordance with the disclosed subject matter.

FIG. 16 provides normalized peak-to-peak displacement images overlaid on the B-mode ultrasound image of orthotropic and mouse tumor at 1, 2, 3, and 4-week post-injection of cancer cell in accordance with the disclosed subject matter.

FIG. 17 provides a graph showing the peak-to-peak displacement ratio of healthy tissue over tumor and tumor diameter as a function of time after tumor cell injection in accordance with the disclosed subject matter.

FIG. 18 provides images Peak-to-Peak (P2P) images overlaid on the B-mode ultrasound image of breast cancer patients with Fibroadenoma (FA), pseudo angiomatous stromal hyperplasia (PASH), and invasive ductal carcinoma (IDC). P2PD ratio of healthy tissue over tumor was 1.37, 1.61, and 1.78 in patients with FA, PASH, and IDC in accordance with the disclosed subject matter.

FIGS. 19A-19D provides images and graphs showing (19A) B-mode ultrasound images of a mouse tumor, (19B) example ST-HMI with multi-frequency excitation pulse induced displacement profile of a pixel in the tumor, (19C) incremental or differential displacement profile, and (19D) Fourier transform of the incremental displacement profile with peaks at 100:100:1000 Hz in accordance with the disclosed subject matter.

FIG. 20 provides images showing example P2PD images at 100:100:1000 Hz overlaid on Bmode image of a mouse tumor at 7^{th} day post-injection of the tumor cells in accordance with the disclosed subject matter.

FIG. 21 provides images showing example P2PD images at 100 Hz overlaid on Bmode image of a mouse tumor at 7, 10, 13, 20, 26, and 32^{nd} day post-injection of the tumor cells in accordance with the disclosed subject matter.

FIG. 22 provides a graph showing a box plot of P2PD in tumor versus oscillation frequency with days (D7 . . . D32) after post-injection of tumor cells in accordance with the disclosed subject matter.

FIG. 23 provides a graph showing the statistical comparison of P2PD at 10, 13, 20, 26, and 32^{nd} day with P2PD at 7^{th} day post-injection of tumor cell in accordance with the disclosed subject matter.

FIG. 24 provides a histology image showing the presence of cancer cells in the liver, indicating diffuse metastases in accordance with the disclosed subject matter.

FIG. 25 provides images showing example P2PD images at 100 Hz overlaid on Bmode image of a mouse liver at 7, 10, 13, 20, 26, and 32^{nd} day post-injection of the tumor cells. P2PD decreased over time in accordance with the disclosed subject matter.

FIG. 26 provides a graph showing a box plot of P2PD in liver versus oscillation frequency with days (D7 . . . D32) after post-injection of tumor cells in accordance with the disclosed subject matter.

FIG. 27 provides graphs showing statistical comparison of P2PD of the liver at 10, 13, 20, 26, and 32^{nd} day with P2PD of the liver at 7^{th} day post-injection of tumor cell in accordance with the disclosed subject matter.

FIG. 28 provides graphs showing linear regression between P2PD displacement at 100, 200, and 400 Hz between tumor versus liver in accordance with the disclosed subject matter.

FIG. 29. provides images showing phase velocity (ms⁻¹) map at 100:100:1000 Hz of homogeneous background region of a commercially available phantom in accordance with the disclosed subject matter.

FIG. 30 provides images showing phase velocity (ms⁻¹) map at 100:100:1000 Hz of 6 kPa inclusion with 6.5 mm diameter embedded in 18 kPa background of a phantom in accordance with the disclosed subject matter.

FIG. 31 provides images showing phase velocity (ms⁻¹) map at 100:100:1000 Hz of 6 kPa inclusion with 10.4 mm diameter embedded in 18 kPa background of a phantom in accordance with the disclosed subject matter.

FIG. 32 provides images showing phase velocity (ms⁻¹) map at 100:100:1000 Hz of 70 kPa inclusion with 6.5 mm diameter embedded in 18 kPa background of a phantom in accordance with the disclosed subject matter.

FIG. 33 provides images showing phase velocity (ms⁻¹) map at 100:100:1000 Hz of 70 kPa inclusion with 10.4 mm embedded in 18 kPa background of a phantom in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for harmonic motion elastography (HME). The disclosed subject matter provides systems and methods for single transducer harmonic motion imaging. The disclosed subject matter provides systems and methods for single transducer harmonic motion imaging induced “on-axis” displacements and “off-axis” shear wave velocity.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

The term “coupled,” as used herein, refers to the connection of a device component to another device component by methods known in the art.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

In certain embodiments, the disclosed subject matter provides a system for single transducer harmonic motion imaging. An example system can include a transducer and a processor. In certain embodiments, the processor can be operatively coupled to the transducer. For example, the processor and the transducer can be coupled directly (e.g., wire connection or installation into the transducer) or indirectly (e.g., wireless connection).

In certain embodiments, the transducer can be used to stimulate the target tissue by generating an acoustic radiation force. For example, the transducer can generate an amplitude-modulated acoustic radiation force (AM-ARF). The AM-ARF can be generated by sinusoidally modulating a duration of an excitation pulse generated by the transducer. In non-limiting embodiments, the transducer can generate a focused beam or unfocused beam. In non-limiting embodiments, the transducer can be a linear array or fully sampled matrix array or array with higher number of lateral versus elevational elements or row-column array or focused ultrasound array.

In certain embodiments, the excitation pulse can have different combinations of parameters. The parameters can include duration, a cycle number, an oscillation frequency, a number of sinusoids, center frequency, F-number, sampling intervals, starting time offset, and/or sample numbers. In non-limiting embodiments, the duration of the discrete excitation pulse can be from about 10 μs to about 150 μs, from about 20 μs to about 150 μs, from about 30 μs to about 150 μs, from about 40 μs to about 150 μs, from about 40 μs to about 140 μs, from about 40 μs to about 130 μs, from about 40 μs to about 120 μs, or from about 40 μs to about 110 μs and/or any combination of minimum duration of about 10 μs and maximum duration of about 300 μs. In non-limiting embodiments, the center frequency of the excitation pulse can range from about 1 megahertz (MHz) to about 20 MHz, from about 1 MHz to about 15 MHz, from about 1 megahertz (MHz) to about 10 MHz, from about 1 megahertz (MHz) to about 5 MHz, from about 1 megahertz (MHz) to about 4 MHz, from about 1 megahertz (MHz) to about 3 MHz, or from about 1 megahertz (MHz) to about 2 MHz and/or 0.5 MHz to about 20 MHz depending on the depth of the target tissue. In non-limiting embodiments, the number of the excitation pulse cycles can be at least about 2, at least about 20, at least about 30, at least about 40, or at least about 50. For example, the excitation pulse can include at least 2 cycles of a 100 Hz signal. In certain embodiments, the parameters of the excitation pulse can be pre-programmed and/or adjusted depending on the target tissue or subject.

In certain embodiments, the excitation pulse can include a sum of a sinusoidal signal. The sinusoidal signal can follow the shape of the sine curve. The sinusoids can have a frequency from about 100 Hz to about 2000 Hz to facilitate data acquisition and/or to generate a displacement map of the corresponding frequency simultaneously.

In certain embodiments, the acoustic radiation force (e.g., AM-ARF) generated by the transducer can be used for inducing harmonic motion on the target tissue. Harmonic motion can occur when the material oscillates around its original location due to a sinusoidally varying force at a specific frequency. For example, the AM-ARF can generate deformation (e.g., the oscillatory motion) from the target tissue. The transducer can generate the harmonic displacement on the target tissue without damaging the target tissue. The displacement can range from about 1 μm to about 40 μm, from about 1 μm to about 30 μm, from about 1 μm to about 20 μm, or from about 1 μm to about 10 μm. In non-limiting embodiments, the transducer can generate AM-ARF-induced displacements at a plurality of frequencies simultaneously. For example, a plurality of frequencies can be generated by using an excitation pulse composed of a sum of sinusoidal signals.

In certain embodiments, the transducer can simultaneously track the harmonic motion by collecting a tracking pulse. The tracking pulse can be interleaved between the excitation pulse for being simultaneously collected by a single transducer.

In certain embodiments, the tracking pulse can have different combinations of parameters. For example, the tracking pulse parameter can include a center frequency, a pulse repetition frequency (PRF), a cycle number, and/or F-number. For example, the center frequency of the tracking pulse can range from about 1 megahertz (MHz) to about 40 MHz, from about 1 MHz to about 30 MHz, from about 1 MHz to about 20 MHz, from about 1 MHz to about 10 MHz, or from about 1 MHz to about 5 MHz. In some embodiments, the center frequency of the tracking pulse can be about 6.1 MHz or 15.6 MHz. In non-limiting embodiments, the number of the tracking pulse cycle can be about 1, about 2, about 3, about 4, or about 5. In non-limiting embodiments, the PRF of the tracking pulse can be from about 5 kHz to about 20 kHz. In certain embodiments, the parameters of the tracking pulse can be pre-programmed and/or adjusted depending on the target tissue or subject.

In certain embodiments, the disclosed system can include a processor coupled to the transducer. The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components described above. For example, and as embodied herein, the processor can be configured to control the operation of the transducer.

In certain embodiments, the processor can be configured to estimate mechanical properties of the target tissue based on the tracked harmonic motion and/or tracking pulse. For example, the processor can generate a displacement map corresponding to the frequencies of the excitation pulse generated by the transducer. In non-limiting embodiments, the mechanical properties can include stiffness, Young's modulus, elasticity, viscosity, porosity, permeability, or combinations thereof.

In certain embodiments, the processor can be configured to generate beamformed radiofrequency (RF) data by performing delay-and-sum beamforming. For example, a custom delay-and-sum beamforming can be applied to the channel data to construct beamformed radiofrequency (RF) data.

In non-limiting embodiments, the processor can be configured to estimate displacements induced by the acoustic radiation (e.g., AM-ARF) based on the RF data. For example, 1-D normalized cross-correlation (NCC) or Loupas or Kasai estimator can be applied to estimate displacement relative to the reference tracking pulse, which can be yielded in a 3-D dataset (e.g., axial×lateral×time) describing axial displacements over time.

In non-limiting embodiments, the processor can be configured to perform a two-dimensional interpolation on the estimated displacements. For example, a 2-D spline interpolation can be applied to the 2-D displacement data at each time point to convert anisotropic pixel size to an isotropic pixel size (e.g., 0.1 mm).

In non-limiting embodiments, the processor can be configured to calculate differential displacements between successive time points based on interpolated displacements. For example, the differential displacements at each pixel can be computed by subtracting displacements between successive time points to remove the slowly varying motion. The differential displacements at each time point can be averaged using a 2-D sliding window (e.g., with a 0.8×0.8 mm kernel). In some embodiments, the spatial averaging of the differential displacements can be performed to reduce noise before filtering out displacement at each frequency.

In non-limiting embodiments, the processor can be configured to filter the differential displacements for target frequencies. For example, the differential displacement profiles can be filtered out using a fourth-order infinite impulse response (IIR) or Finite impulse response (FIR), or Butterworth bandpass filter to estimate displacements at each frequency. In some embodiments, filtering of differential displacement profiles can be performed separately at each frequency, and at each pixel, the cutoff values of the bandpass filter can be selected adaptively.

In non-limiting embodiments, the processor can be configured to generate a peak-to-peak displacement (P2PD) image based on the filtered differential displacements. For example, the filtered displacement profile at each pixel and each frequency can be integrated and normalized to a zero mean. Using the integrated-filtered displacement profile, the average P2PD can be calculated at each pixel, and then, rendered into a 2-D parametric image. In some embodiments, the P2PD images at each frequency can be normalized separately to account for the variation in the acoustic radio force magnitude over the axial range.

In certain embodiments, the processor can be configured to generate a phase velocity map corresponding to the frequencies of the excitation pulse. In non-limiting embodiments, the processor can be configured to generate beamformed radiofrequency (RF) data by performing delay-and-sum beamforming, estimate displacements induced by the AM-ARF based on the RF data, perform a two-dimensional interpolation on the estimated displacements, calculate differential displacements between successive time points based on interpolated displacements, apply directional filtering, apply one dimension Fourier transform along the time dimension to select frequencies of the excitation pulse, apply two-dimensional Fourier transform along spatial dimension at the selected frequency and calculate phase velocity at the selected frequency using the relationship between wavenumber and temporal frequency or linear regression of phase versus distance.

In certain embodiments, the processor can be configured to generate quantitative mechanical properties (e.g., viscosity, porosity, permeability, and elasticity) by fitting phase velocity versus frequency relationship derived analytically or empirically using rheological models.

In certain embodiments, phase velocities at multiple frequencies can be generated at two orthogonal directions by mechanically rotating a linear array transducer or electronically rotating point spread function using a matrix array or a transducer with more than one element in an elevational direction or a row-column array (e.g., a 1.5D or 2D array), and phase velocities at the orthogonal directions can be used to derive a degree of anisotropy as a function of frequency.

In certain embodiments, the target tissue can be any tissue. For example, the target tissue can be a cancer, a tumor, a nerve, a brain, a heart, muscle, tendons, ligaments, skin, vessels, artery, tumor, cancer, prostate tissue, arterial tissue, renal tissue, or a combination thereof.

In certain embodiments, the disclosed subject matter provides a method for single transducer harmonic motion imaging. An example method can include generating an amplitude-modulated acoustic radiation force (AM-ARF) by sinusoidally modulating a duration of an excitation pulse of an acoustic radiation force, inducing a harmonic motion on a target tissue using the AM-ARF, simultaneously tracking the harmonic motion by collecting a tracking pulse and estimating the mechanical properties of the target tissue based on the tracked harmonic motion. In non-limiting embodiments, the tracking pulse can be interleaved between the excitation pulse. In some embodiments, the disclosed method can be performed using the disclosed system, excitation pulse, tracking pulse, transducer, and/or processor.

In certain embodiments, the method can further include generating beamformed radiofrequency (RF) data by performing delay-and-sum beamforming, estimating displacements induced by the AM-ARF based on the RF data, performing a two-dimensional interpolation on the estimated displacements, calculating differential displacements between successive time points based on interpolated displacements, filtering the differential displacements for target frequencies, generating peak-to-peak displacement (P2PD) image based on the filtered differential displacements. In non-limiting embodiments, the disclosed method can be performed using the disclosed system and transducer. In non-limiting embodiments, the disclosed method can be performed using the disclosed system, excitation pulse, tracking pulse, transducer, and/or processor.

In certain embodiments, the method can include generating AM-ARF-induced displacements at a plurality of frequencies simultaneously. In non-limiting embodiments, the method can further include generating a displacement map corresponding to the frequencies of the excitation pulse. For example, the excitation pulse can include a sum of sinusoids with about 100-about 1000 Hz frequencies.

In certain embodiments, the method can include determining a metastatic location and a metastatic level of a subject based on the estimated mechanical properties. The mechanical properties can include stiffness, shear modulus, Young's modulus, elasticity, viscosity, or combinations thereof. In some embodiments, various target tissues can be assessed to determine the metastatic location and level. For example, the target tissue can include cancer, tumor, brain tissue, liver tissue, pancreatic tissue, breast tissue, prostate tissue, heart tissue, renal tissue, and combinations thereof. The stiffness of the metastatic location can be higher than surrounding healthy tissue. By measuring P2PD or phase velocity, the location of the metastatic location can be detected. Stiffness of the metastatic location can also be used to monitor the response of the treatment. In non-limiting embodiments, the method can further include administering a treatment to the metastatic location based on the metastatic level. For example, additional treatments can be administered into a region with a higher metastatic level. The treatment can include anti-tumor treatment, anti-cancer treatment, chemotherapy, immunotherapy, radiation, surgery, or combinations thereof.

In certain embodiments, the disclosed subject matter provides techniques for generating a phase velocity map corresponding to the frequencies of the excitation pulse. Phase velocity denotes the speed of the resulting shear wave phase at a particular frequency. The stiffer tissue will have higher phase velocity than softer tissue. Phase velocity will be calculated at each pixel and image of phase velocity map will be generated. An example method can include generating an amplitude-modulated acoustic radiation force (AM-ARF) by sinusoidally modulating a duration of an excitation pulse of an acoustic radiation force, inducing shear waves at one or more frequencies simultaneously on a target tissue using the AM-ARF, tracking the shear wave by collecting a tracking plane wave frames at a plurality angles with a frame rate between about 5 to about 20 kHz or collecting a focused pulse at a pulse repetition frequency of from about 5 to about 20 kHz between the excitation pulses, generating a phase velocity map based on the tracked shear wave, and estimating mechanical properties of the target tissue based on the phase velocity map. In non-limiting embodiments, the excitation pulse can include at least approximately 2 cycles of the lowest harmonic frequency. The disclosed system can be used for generating the phase velocity map.

In certain embodiments, one dimension and/or two-dimension Fourier transformation can be applied to the displacement data obtained by the disclosed subject matter. For example, the method can further include generating beamformed radiofrequency (RF) data by performing delay-and-sum beamforming, estimating displacements induced by the AM-ARF based on the RF data, performing a two-dimensional interpolation on the estimated displacements, calculating differential displacements between successive time points based on interpolated displacements, perform filtering on the calculated differential displacements to remove reflected shear waves, performing one dimensional Fourier transformation on the filtered differential displacements along time dimension to select a frequency of the excitation pulse, performing two-dimensional Fourier transform on the transformed differential displacements along spatial dimension at the selected frequency of the excitation pulse, and calculating a phase velocity at the selected frequency based on properties of the time and spatially Fourier transformed differential displacements.

In certain embodiments, the properties of the temporally and spatially Fourier transformed differential displacements can include a wave number, a temporal frequency, a linear regression of phase versus distance. or combinations thereof.

In certain embodiments, the method can include generating quantitative mechanical properties of the target tissue by using P2PD versus frequency or phase velocity versus frequency relationship of by using a rheological model. In non-limiting embodiments, a rheological model with quantitative mechanical properties like viscosity and elasticity can be Maxwell Model, Kelvin-Voigt model, standard linear solid model, Burgers model, generalized Maxwell model or Prony series. There are analytical equation describing relationship between phase velocity versus frequency. The relationship between P2PD versus frequency can be derived empirically or analytically for these rheological models. The least minimization will be performed between the estimated versus analytical or empirical relationship of P2PD versus frequency or phase velocity versus frequency.

In certain embodiments, the shear waves at one or more frequencies can be generated at two orthogonal directions by mechanically rotating a linear array transducer or electronically rotating point spread function using a 1.5D or matrix array. In non-limiting embodiments, the shear waves at the orthogonal directions can be used to derive a degree of anisotropy as a function of frequency.

In certain embodiments, the phase velocity map can be used for determining a metastatic location and a metastatic level based on the estimated mechanical properties and administering a treatment to the metastatic location based on the metastatic level.

Phase velocity methods provide quantitative mechanical properties like elasticity and viscosity. The shear wave based methods can suffer from artifacts emanating from tissue heterogeneities and reflected shear waves, poor mechanical resolution, and lower penetration depth. Displacement map can provide qualitative mechanical properties but have better mechanical resolution and higher penetration depth than phase velocity map. In certain embodiments, the disclosed phase velocity map can directly be linked to the underlying stiffness (e.g., albeit with several artifacts) whereas the displacement provides a relative stiffness measurement.

### EXAMPLES

### Example 1: Imaging of Single Transducer-Harmonic Motion Imaging-Derived Displacements at Several Oscillation Frequencies Simultaneously

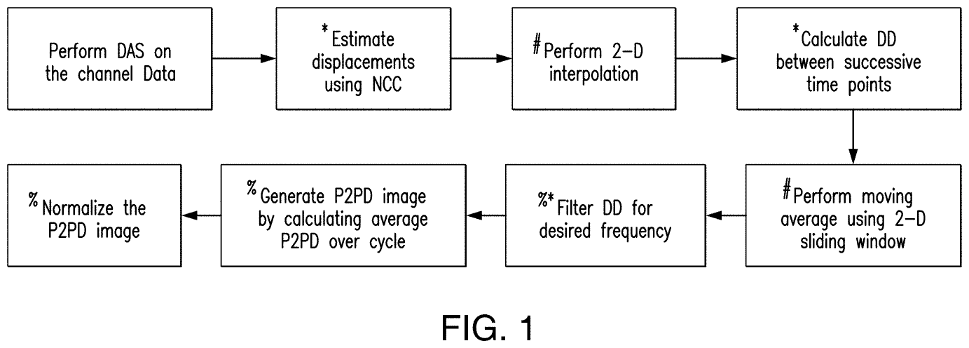

Single transducer-harmonic motion imaging (ST-HMI) can interrogate mechanical properties of tissues “on-axis” to the acoustic radiation force (ARF) by generating harmonic motion (HM) at a particular frequency via transmitting tracking pulses in-between discrete excitation pulses. To facilitate data acquisition, the disclosed subject matter provides a new excitation pulse composed of a sum of sinusoids with 100-1000 Hz frequencies to generate a displacement map of the corresponding frequency simultaneously. The performance of the proposed excitation pulse was assessed by imaging 16 different inclusions (e.g., Young's moduli of 6, 9, 36, 70 kPa and diameters of 1.6, 2.5, 6.5, and 10.4 mm) embedded in an 18 kPa background and is compared to the ARFI imaging. Depending on inclusion size and stiffness, the maximum Contrast and contrast to noise ratio (CNR) and contrast were achieved at different frequencies and were always higher than ARFI. The frequency, where maximum CNR and contrast were achieved, increased with increasing stiffnesses for fixed inclusion's size and increased with decreasing size for fixed stiffness. As shown in FIG. 1, the peak-to-peak displacement (P2PD) ratio of background over inclusion was highly correlated with Young's moduli ratio with R2≥0.9 irrespective of frequencies or inclusion sizes. In vivo feasibility is tested by imaging tumors in a mouse cancer model (N=2) after 3-days of cancer cell implantation. The maximum P2PD ratio was achieved at 900 and 800 Hz frequency in the two animals tested. These results indicate the importance of using a multi-frequency excitation pulse to simultaneously generate displacement at multiple frequencies to better delineate inclusions or lesions.

Excitation Pulse Composed of Sum of Sinusoids: The disclosed multi-frequency excitation pulse was composed of a sum of sinusoids with the lowest frequency of f_L and was generated as follows:

$${e}_{1} ( t ) = {\sum }_{j = 1}^{{N}_{s i n u s o i d}} {j}^{2} \times cos ( 2 π j {f}_{L} t + {θ}_{h} ) , where , \\ {θ}_{j} = \{ \begin{matrix}0 , if j odd \\ π , if j even\end{matrix} \qquad ( 1 )$$

where N_{sinusoid} defines the total number of sinusoids with a frequency of an integer multiple of f_{L}. Therefore, the maximum frequency in e₁(t) is N_{sinusoid}×f_{L}. The duration of the excitation pulse is the product of cycle number (N_{cycle}) and fundamental period T_{L}=1/f_{L}. The multiplication term, j² in (1) is added to account for the higher loss in the higher frequencies. As e₁(t) contains both positive and negative values, a constant, A_{offset}, is added to have the excitation pulse with only positive values.

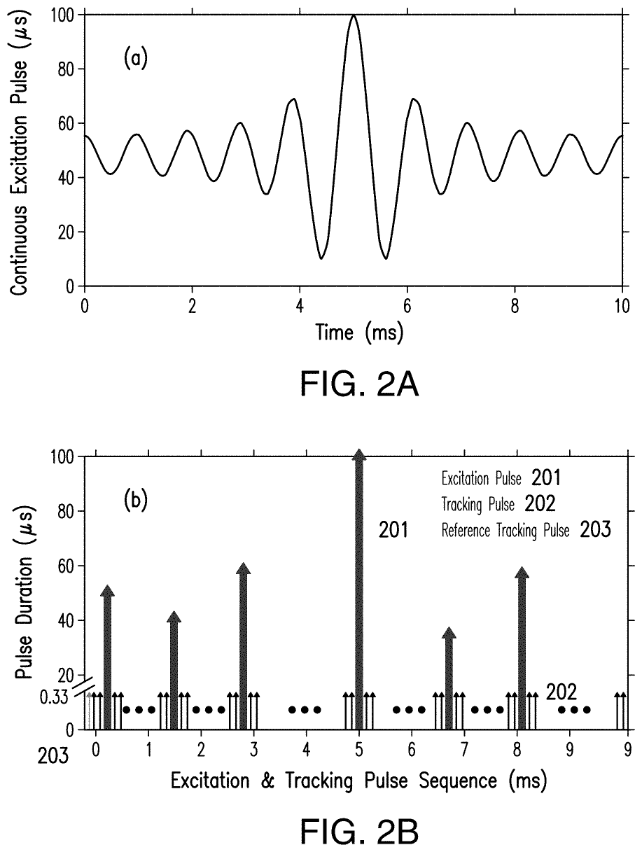

e₂(t)=A_{offset}+e₁(t),where, A_{offset}=−A_{factor}×min(e₁(t)) (2)

where min(e₁(t)) means minimum of e₁(t). A_{factor} in (2) defines the minimum excitation pulse duration. Therefore, A_{factor}=1.0 means the minimum value of e₂(t) is zero. Finally, e₂(t) is normalized as follows to have a maximum excitation pulse duration of t_{ARF}^{max}.

$$e ( t ) = \frac{{t}_{A R F}^{max} \times {e}_{2} ( t )}{max ( {e}_{2} ( t ) )} \qquad ( 3 )$$

where, max(e₂(t)) means maximum of e₂(t). However, e(t) in (3) is a continuous excitation pulse (see FIG. 2A). To accommodate both excitation and tracking pulses, e(t) is sampled to generate Nₑₚ discrete excitation pulses as follows:

E[n]=e(t)×Σₙ^{N}^{ₑₚ}δ(t−tₙ) (4)

the δ is the Delta-Dirac function and to defines the n^{th} discrete excitation pulse's location in the time-axis. Tracking pulses are interleaved between Nₑₚ discrete excitation pulses 201 (see FIG. 2B). The induced displacement was estimated relative to the reference tracking 203 pulse, which was transmitted at the start of the excitation and tracking pulse sequence 202.

Phantom Experiments: The feasibility of generating displacements at multi-frequencies simultaneously was tested by imaging a commercially available elastic [symbol] phantom (model 049A, CIRS, Norfolk, VA, USA). The imaging was performed using a Verasonics research system (Vantage 256, Verasonics Inc., Kirkland, Wash., USA) equipped with an L7-4 transducer (Philips Healthcare, Andover, Mass., USA). Using a clamp, the transducer was held in a steady position. Four stepped-cylindrical inclusions with nominal Young's moduli of 6, 9, 36, and 70 kPa were embedded in the background with nominal Young's modulus of 18 kPa. For each stiffness, imaging was performed at cross-sections with 1.6, 2.5, 6.5, 10.4 mm diameters. The manufacturer provided standard deviation in elasticity and diameters measurements was approximately 5%. The center of the inclusion was approximately 30 mm from the phantom's surface. However, water was added between the transducer's and phantom's surface, which resulted in the center of inclusion at 34 mm from the transducer surface. Throughout the remainder of the manuscript, each inclusion can be represented by its mean nominal Young's modulus and diameter.

TABLE 1
Example parameters of ARFU and ST-HMI

[symbol]

| | | |
|---|---|---|
| Parameters | Phantom | Mouse |

Beam sequence parameters of ST-HMI/ARFI

| Transducer | 1.7-4 | | 1.22- | |
|---|---|---|---|---|
| | | | 14vXLF | |
| Bandwidth | 58% | | 51% | |
| Sampling frequency | 20.84 | MHz | 62.5 | MHz |
| Acoustic lens axial focus | 25 | mm | 20 | mm |
| Excitation pulse center frequency | 4.0 | MHz | 15.6 | MHz |
| Excitation pulse F-number | 2.25 | | 2.25 | |
| Tracking pulse center frequency | 6.1 | MHz | 20.8 | MHz |
| Tracking pulse receive F-number* | 1.0 | | 1.0 | |
| Excitation and tracking pulse axial focus | 34 | mm | 14 | mm |
| Spacing between RF-lines | 0.59/0.3 | mm | 0.3 | mm |
| RF-lines number/image | 32/38 | | 22 | |
| Lateral field of view size | 20/11 | mm | 6.3 | mm |
| Tracking pulse PRF | 10 | KHz | 15 | KHz |

ST-HMI specific parameters

| Lowest oscillation frequency, f [symbol] | 100 | Hz | 100 | Hz |
|---|---|---|---|---|
| [symbol] number, N [symbol] | 10 | | 10 | |
| [symbol] | 1.25 | | 1.25 | |
| Maximumum excitation pulse | 100 | μs | 40 | μs |
| duration [symbol] | | | | |

| | | |
|---|---|---|
| Cycle number, N_{cycle} | 6 | 5 |

ARFI Specific parameters

| Tracking pulse number | 110 | | — |
|---|---|---|---|
| Excitation pulse duration | 113 | μs | — |

Normalized cross correlation parameter

| Interpolation factor | 4 | | 4 | |
|---|---|---|---|---|
| Kernel length | 592 | μm | 492 | μm |
| Search region | 80 | μm | 80 | μm |

*Aperature growth and dynamic Rx focusing enabled [symbol]
indicates data missing or illegible when filed

The performance of ST-HMI with multi-frequency excitation pulse was compared to ARFI imaging. The ARFI and ST-HMI imaging were performed consecutively with parameters indicated in Table I. Briefly, both ARFI and ST-HMI data were collected using focused excitation and tracking beams generated with sub-aperture and translated electronically across the lateral field to generate a 2-D image. Thirty-two or Thirty-eight evenly spaced RF lines with 0.6 mm or 0.3 mm spacing between RF lines were acquired to image inclusions with diameters of (10.4 and 6.5 mm) or (2.5 and 1.6 mm), respectively. Wiper blading scanning mode was used to prevent interference in the tissue mechanical response between consecutive RF lines and reduce transducer face heating. One frame of the B-mode ultrasound image with 128 RF lines spanning approximately 38 mm in lateral direction was collected preceding ARFI and ST-HMI imaging. By moving the transducer in the elevational direction, six repeated acquisitions of ARFI and ST-HMI were acquired at each inclusion stiffness and size. The acquisition time of ST-HMI data with 32 RF lines took approximately 6 s with 0.1 s interval between RF lines.

Imaging of A breast cancer mouse model, In Vivo: The in vivo performance of the proposed excitation pulse sequence was investigated by imaging tumors in an orthotropic, 4T1 breast cancer mouse model (N=2). The Columbia University Irving Medical Center (CUIMC) Institutional Animal Care and Use Committee (IACUC) reviewed and approved the protocol for the cancer induction and imaging of the mouse's tumors. Tumors were generated by injecting 1×10⁵ 4T1 breast cancer cells in the 4^{th} inguinal mammary fat pad of the eight to ten-week-old female BALB/c mice (Jackson Laboratory).

The same Vantage Verasonics research system equipped with L22-14vXLF (Vermon, Tours, France) linear array was used to perform ST-HMI. Briefly, the anesthetized mice (1-2% isoflurane in oxygen) were imaged by placing the mice in a supine position on a heating pad with their abdominal hair removed, and the transducer was held in a steady position using a clamp during imaging. Both mice were imaged at 3-day post-injection of cancer cell using the parameters indicated in Table I. Twenty-two evenly spaced RF-lines with 0.3 mm separation in between RF-lines were acquired to generate a 2-D image of ST-HMI-derived P2PD. Preceding each ST-HMI sequence, one spatially-matched B-mode image was acquired with 128 lateral lines spanning approximately 13.6 mm, for anatomical reference.

ST-HMI and ARFI Data Processing: The channel data were stored onto the Verasonics workstation after running ARFI and ST-HMI imaging sequence and were transferred to the computational workstation for offline processing using MATLAB (MathWorks Inc., Natick, Mass., USA). A custom delay-and-sum beamforming was applied to the channel data to construct beamformed radiofrequency (RF) data. 1-D normalized cross-correlation (NCC) (Table I) was applied to estimate displacement relative to the reference tracking pulse, which yielded in a 3-D dataset (axial×lateral×time) describing axial displacements over time.

From the ARFI 3-D dataset, a parametric 2-D image of PD was generated after applying a linear filter to the displacement versus time profile at each pixel. Finally, ARFI-derived PD images were normalized to account for the variation in the ARF magnitude over the axial range. A 2-D spline interpolation (interp2 function) was applied on the normalized PD image to convert anisotropic pixel dimension (0.04×0.6 mm or 0.04×0.3 mm) to an isotropic pixel dimension of 0.1 mm.

2-D parametric image of ST-HMI-derived P2PD at each frequency was generated. First, a 2-D spline interpolation (interp2 function) was applied on the 2-D displacement data at each time point to convert anisotropic pixel size to an isotropic pixel size of 0.1 mm. Second, the differential displacements at each pixel were computed by subtracting displacements between successive time points to remove the slowly varying motion. Third, the differential displacements at each time point were averaged using a 2-D sliding window with a 0.8×0.8 mm kernel. Note, the differential displacements calculation can act as a high pass filter and has the potential to enhance noise. Therefore, the spatial averaging of the differential displacements was performed to reduce noise before filtering out displacement at each frequency. Fourth, the differential displacement profiles were filtered out using a fourth-order infinite impulse response (IIR) bandpass filter (designfilt and filter function) to estimate displacements at each frequency. It is noteworthy to mention that filtering of differential displacement profiles was performed separately at each frequency. At each pixel, the cutoff values of the bandpass filter were selected adaptively. Fifth, the filtered displacement profile at each pixel and each frequency were integrated (cumsum function in MATLAB) and normalized to a zero mean. Sixth, using the integrated-filtered displacement profile, the average P2PD over N_{cycle}_{^{f}} cycles was calculated at each pixel, and then, rendered into a 2-D parametric image. Note, N_{cycle}_{^{f}} varies between frequencies, i.e., 100 and 1000 Hz had 6 cycles and 60 cycles of oscillation, respectively (Table I). Seventh, P2PD images at each frequency were normalized separately to account for the variation in the ARF magnitude over the axial range. The normalizing profiles for both ARFI and ST-HMI were generated from the 1.5 mm leftmost and rightmost lateral field of view (FOV). FIG. 1 depicts a flowchart representing the processing implemented to generate normalized P2PD images at each frequency.

It took 5 min to process data from performing the delay-and-sum beamforming to generating the final normalized P2PD image at each frequency using a 2.2 GHz Intel Xeon Platinum processor with a 20 cores processor. The computational time can be reduced by implementing ST-HMI data processing pipelines (FIG. 1) in CUDA GPU.

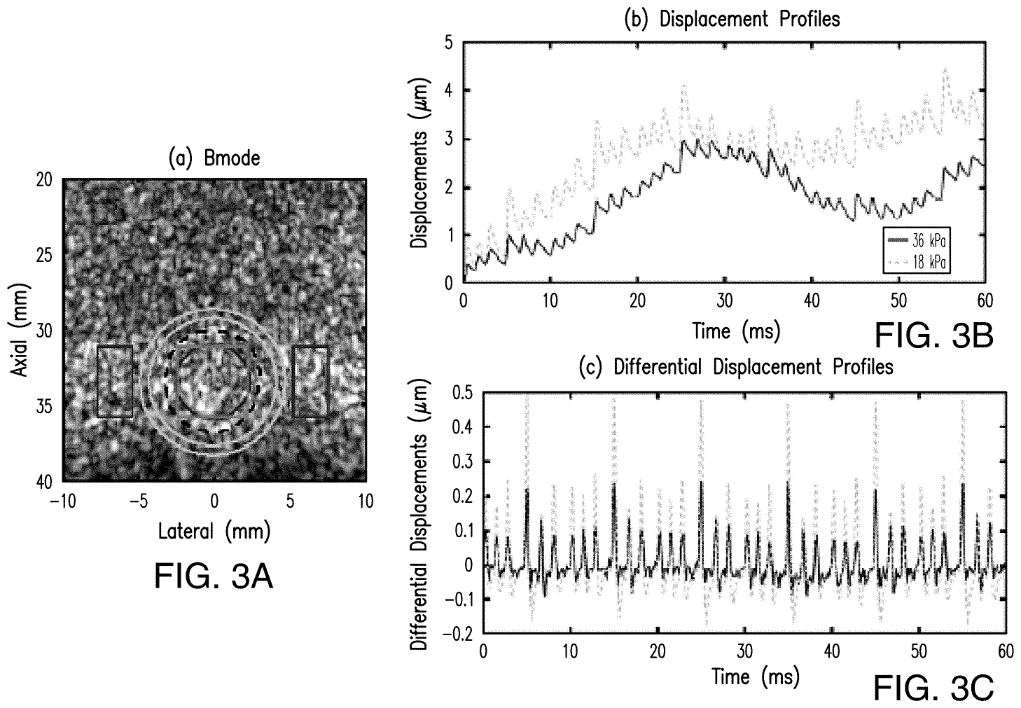

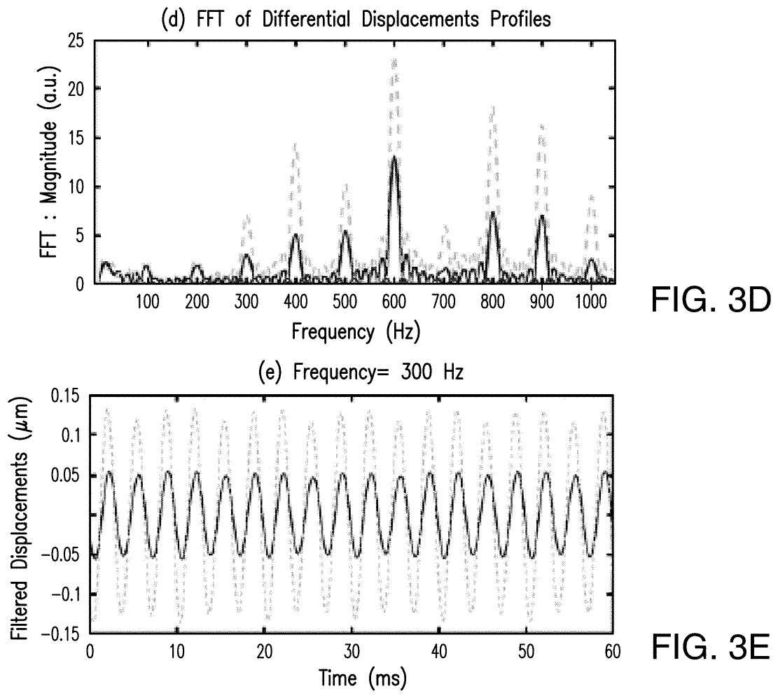

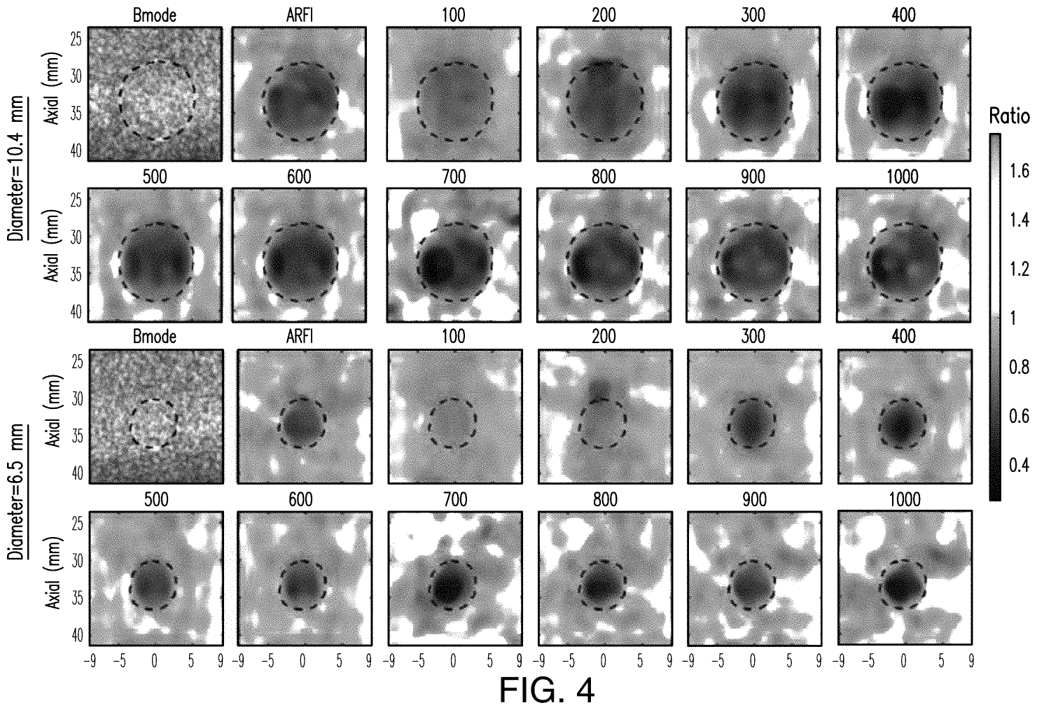

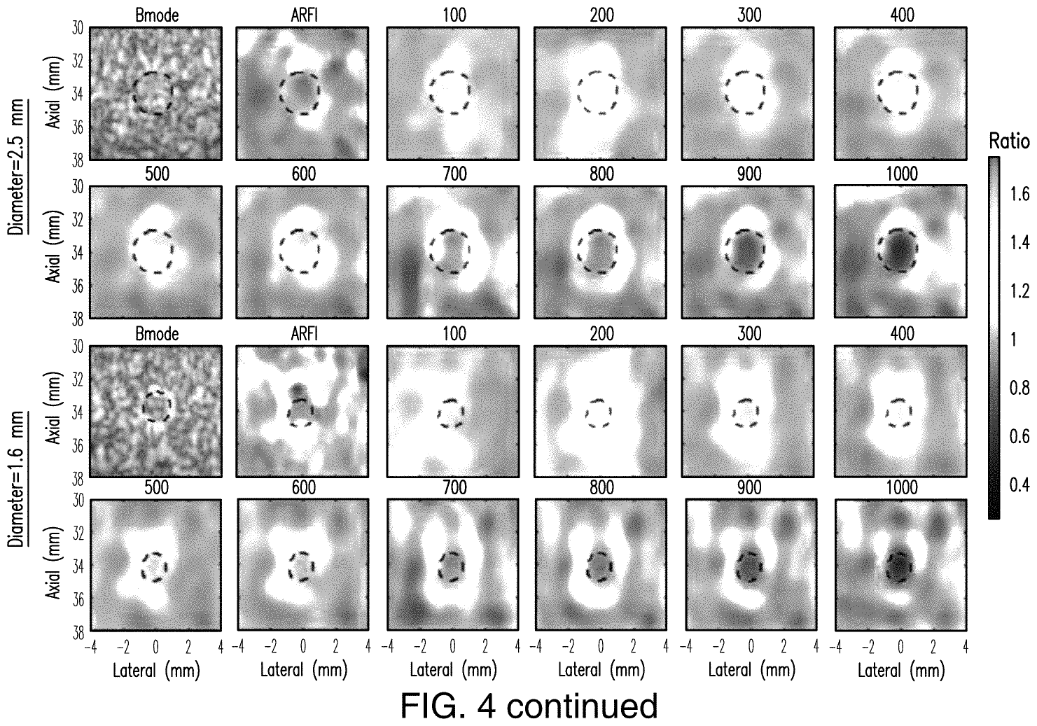

Image Quality Metrics: The performance of ARFI-derived PD and ST-HMI-derived P2PD images were compared quantitatively in terms of contrast and CNR with the region of interests (ROIs) in inclusion (INC) and background (BKD) as the concentric circle and ring, respectively (see FIG. 3A). Contrast and CNR were computed as |μ_{INC}−μ_{BKD}|/μ_{BKD} and |μ_{INC}−μ_{BKD}|/\sqrt{(σ_{INC}²+σ_{BKD}²)}, respectively, where μ and σ are the median and standard deviation of normalized displacements in the ROI. To perform linear regression between P2PD ratios versus Young's moduli ratios, a rectangular ROI (see FIG. 3A) was used to avoid the boundary effects. The inclusion's boundary was derived from the B-mode image (see FIGS. 3A and 4).

Statistical Analysis: All statistical analyses were performed in MATLAB. Thirty-two (diameter, N=4, stiffness, N=4) separate Kruskal-Wallis tests (kruskalwallis function), were carried out to compare the contrast and CNR of ARFI-derived PD and ST-HMI derived P2PD images at 100:100:1000 Hz. If any group was statistically significant, a two-sample Wilcoxon signed rank-sum test (signrank function) was used to find which combination was statistically significant. The R² of the linear regression between the PD or P2PD ratio versus Young's moduli ratio was calculated at each frequency and inclusion size. For all the analyses, the statistical significance was based on α<0.05.

RESULTS: FIG. 2A shows multi-frequency continuous excitation pulse, e(t) (equation (3)) with f_{L}=100 Hz, N_{sinusoid}=10, and N_{cycle}=1 of f_{L}. Therefore, the continuous excitation pulse mainly contains frequencies from 100 to 1000 Hz in steps of 100 Hz. From here onward, 100:100:1000 Hz can represent frequencies from 100 to 1000 Hz in steps of 100 Hz. The continuous excitation pulse was sampled to accommodate both tracking and discrete excitation pulses (FIG. 2B). Note, there were only 6 discrete excitation pulses (Nₑₚ=6) per one-cycle of f_{L}. The duration of the discrete excitation pulses was variable, but the tracking pulse duration was fixed to 0.33 μs (i.e., 2 cycles of 6 MHz). The number of the tracking pulses in between the excitation pulses depends on the pulse repetition frequency (PRF) of the tracking pulse (Table I).

FIGS. 3A-3E shows a representative B-mode ultrasound image of a 6.5 mm, 36 kPa inclusion embedded in an 18 kPa background (FIG. 3A) and representative displacement profiles in inclusion and background with frequency spectrum (FIGS. 3B-3E). The inclusion's boundary was derived from the B-mode and was used to draw ROI for contrast and CNR calculation. Displacements (FIG. 3B) or differential displacements (FIG. 3C) were higher in 18 kPa versus 36 kPa material which is expected. Six peaks per period (10 ms) correspond to the six discrete excitation pulses (FIG. 2B). The amplitude of each peak was different due to the difference in the duration of the discrete excitation pulse. The Fourier transform of the differential displacements (FIG. 3D) contains peaks at 100:100:1000 Hz. These indicate that the multi-frequency excitation pulse with peaks at 100:100:1000 Hz generated displacements with peaks at 100:100:1000 Hz. FIG. 3E shows displacements at 300 Hz after applying Bandpass filtering with [283 315] Hz cutoff values to the differential displacement profiles. The P2PD was 0.11 and 0.27 μm in 36 and 18 kPa materials at 300 Hz. Similar to FIG. 3E, P2PDs were calculated for each pixel and each frequency to generate corresponding frequency P2PD images.

FIG. 4 shows representative ARFI PD image and ST-HMI P2PD images at 100:100:1000 Hz of 36 kPa inclusion with 10.4, 6.5, 2.5, and 1.6 mm diameters. Note, all images were normalized to account for the variation in the ARF magnitude over axial distance. Four observations are notable. First, the perceived contrast of inclusion varies with the inclusion size for the fixed 36 kPa stiffness irrespective of ARFI or ST-HMI. Second, qualitatively ARFI contrasted 10.4 and 6.5 mm inclusions but was unable to contrast 2.5 or 1.6 mm inclusion. Third, ST-HMI contrasted all inclusions, and the perceived contrast varied with the frequency. This result indicates that the frequency in ST-HMI can be exploited to detect different size inclusions with the same stiffness. Fourth, the number of frequencies contrasted with inclusions decreases with size. As an example, all frequencies contrasted 10.4 mm inclusion, whereas only 900 and 1000 Hz contrasted 1.6 mm inclusion.

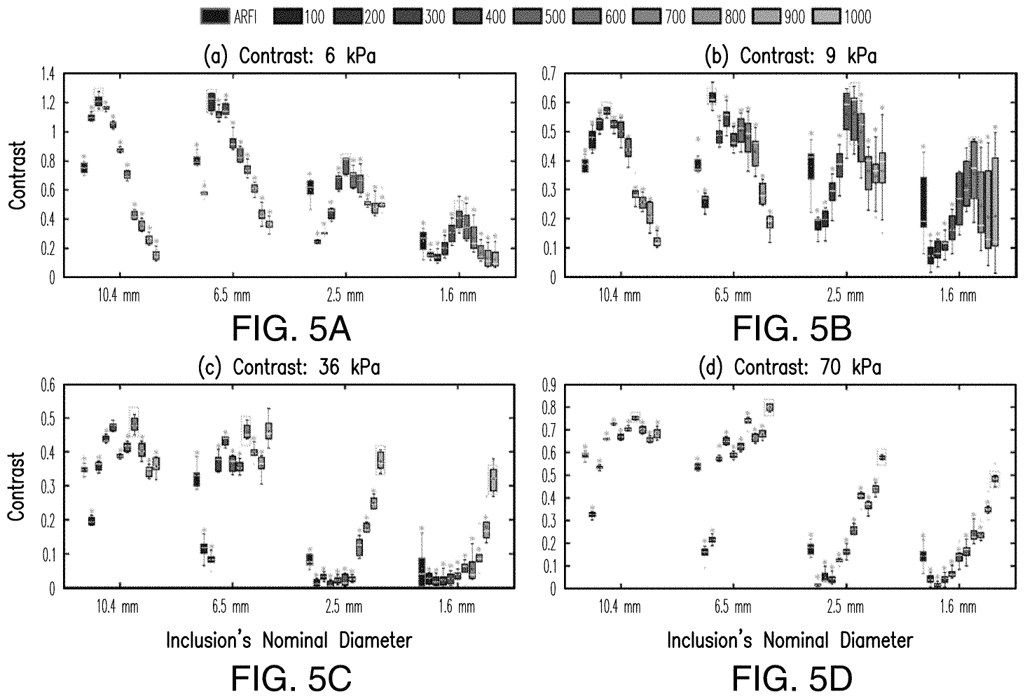

FIGS. 5A-5D quantitatively compare between ARFI versus ST-HMI derived contrast of 6 kPa (FIG. 5A), 9 kPa (FIG. 5B)), 36 kPa (FIG. 5C), and 70 kPa (FIG. 5D) inclusions with 10.4, 6.5, 2.5, and 1.6 mm diameters. Five observations are notable. First, the contrast was statistically different (p<0.05, kruskalwallis test) between ARFI and ST-HMI at 100:100:1000 Hz irrespective of inclusion sizes or stiffnesses. Second, the frequency of ST-HMI can be exploited to achieve higher contrast (p<0.05, ranksum test) than ARFI. Third, the maximum contrast depends on the inclusion size and stiffness. Fourth, for fixed stiffness, maximum contrast decreases with inclusion size. Fifth, the frequency at which the maximum contrast was achieved also depended on the inclusion stiffness and size. The maximum contrast was achieved at (200, 200, 500, 500), (300, 200, 500, 700), (600, 700, 1000, 1000), and (700, 1000, 100, 1000) Hz frequency for 6, 9, 36, and 70 kPa inclusions with (10.4, 6.5, 2.5, 1.6) mm diameters, respectively.

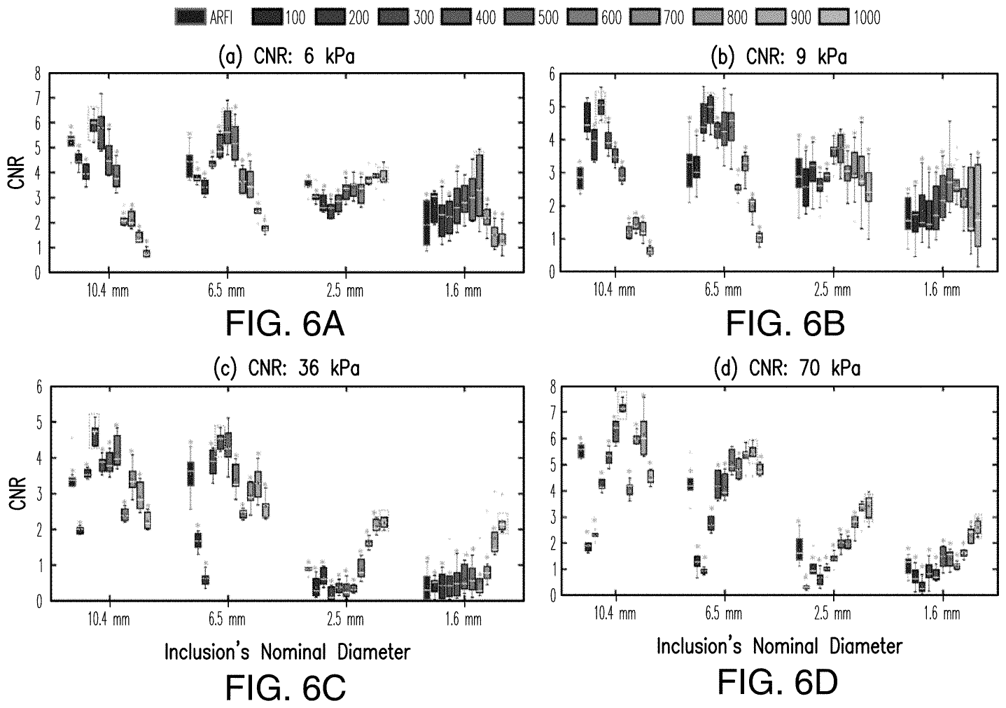

FIGS. 6A-6D quantitatively compare between ARFI versus ST-HMI derived CNR of 6 kPa (FIG. 6A), 9 kPa (FIG. 6B), 36 kPa (FIG. 6C), and 70 kPa (FIG. 6D) inclusions with 10.4, 6.5, 2.5, and 1.6 mm diameters. Observations similar to the contrast in FIG. 5 can be made, i.e., frequency of ST-HMI can be exploited to achieve higher CNR than ARFI, and maximum CNR depends on frequency and inclusion size, and stiffness. However, the frequencies at which the maximum CNR was achieved were different from those at the maximum contrast. The maximum CNR was at achieved (300,500,900, 700), (300, 300, 600, 600), (300, 400, 1000, 1000), and (600, 900, 1000, 1000) Hz frequency for 6, 9, 36, and 70 kPa inclusions with (10.4, 6.5, 2.5, 1.6) mm diameters, respectively. Note, only median values versus median and standard deviation were used in contrast versus CNR calculation. Therefore, CNR accounts for the heterogeneity of background and inclusion. CNR greater than 1 is needed to reliably detect inclusion.

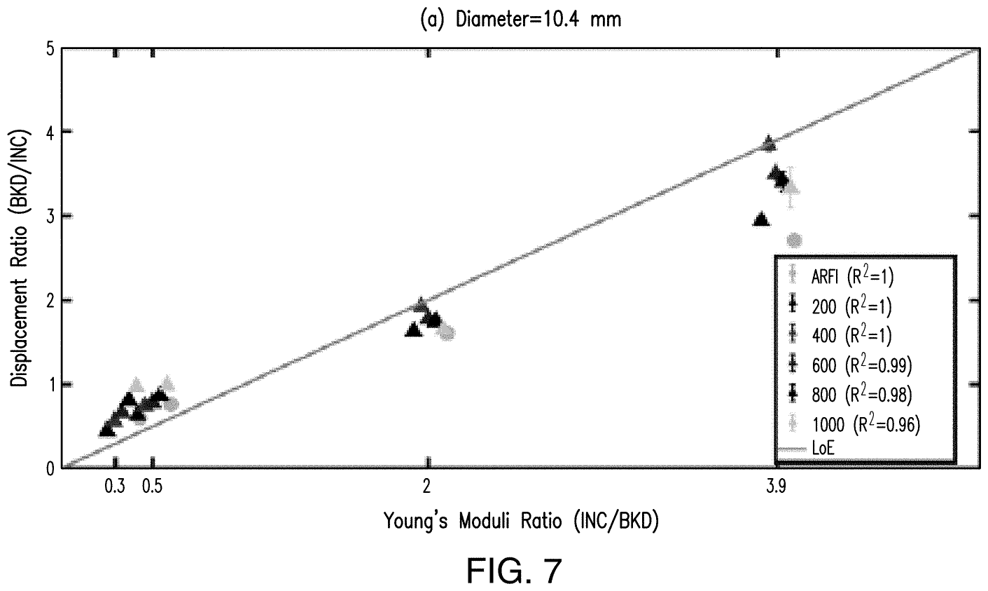

FIG. 7 shows linear regression between ARFI PD ratio or ST-HMI P2PD ratio of background over inclusion versus Young's moduli ratio of inclusion over the background with R² values for 10.4 mm. The R² values were high irrespective of ARFI or ST-HMI with the lowest R² value of 0.96 for 1000 Hz. The R² value and the range in R² value for ARFI PD and ST-HMI P2PD were (1.0, 0.99, 0.99) and (0.93-1.0, 0.91-1.0, 0.93-1.0) respectively for (6.5, 2.5, 1.6) mm diameters, respectively.

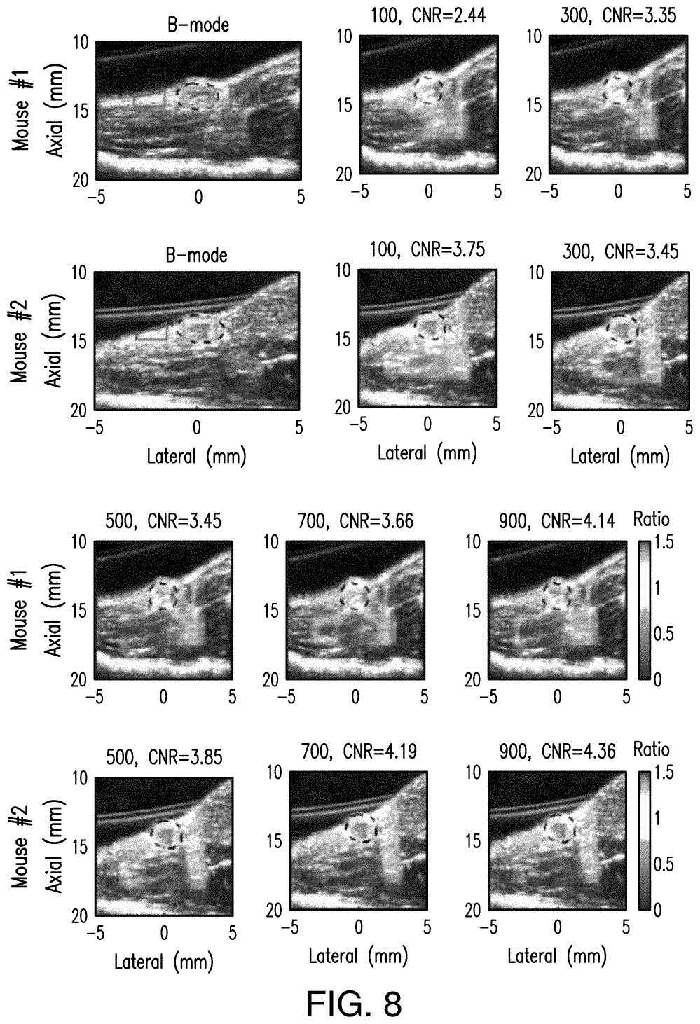

FIG. 8 shows B-mode ultrasound and P2PD images at 100:200:900 Hz overlaid on the B-mode image in both mice with corresponding CNR at the title. The diameter of the tumor was 2.2 and 2.4 mm for mouse #1 and 2, respectively. Two observations are notable. First, all frequencies detected tumors for mouse #1 and 2, respectively. Second, similar to phantom, CNR or the P2PD ratio of healthy tissue to tumor depends on frequencies. The median±0.5*IQR values of (CNR, P2PD ratio) was (3.54±0.55, 3.21±0.73) and (3.76±0.2, 4.2±0.26), for mouse #1 and 2, respectively. The maximum (CNR, P2PD ratio) was achieved at (900, 900) and (900, 800) Hz for mouse #1 and #2, respectively.

Conventional HMI uses AM-ARF to interrogate mechanical properties by oscillating tissue at a particular frequency. To do so, HMI simultaneously generates and tracks harmonic oscillation with a frequency less than 100 Hz using focused ultrasound and imaging transducers, respectively. To facilitate data acquisition, ST-HMI can be used to generate ST-HMI-induced oscillation in the range of 60-420 Hz. However, all frequencies were collected separately. Though oscillation frequency can be exploited to better detect inclusions/lesions, acquisition of multiple frequencies separately can be unrealistic in clinical settings due to patients' or sonographer hand movements. To facilitate the generation of displacement maps at several frequencies, this work presents a novel excitation pulse for ST-HMI to collect multi-frequency data simultaneously. The novel excitation pulse consists of a sum of sinusoids with 100:100:1000 Hz frequencies. The initial feasibility of generating P2PD images at 100:100:1000 Hz was demonstrated in phantoms and in a breast cancer mouse model, in vivo.

ST-HMI assesses mechanical properties “on-axis” to the ARF and is different from the “off-axis” shear wave-based methods like supersonic shear imaging, shear wave imaging (SWI), shearwave dispersion ultrasound vibrometry, or harmonic SWI in terms of estimating the mechanical properties of tissues. Though an excitation pulse composed of a sum of sinusoids was used in shear wave-based methods, there are several differences. Unlike other techniques, the disclosed subject matter can provide the advantages of assessing mechanical properties “on-axis” to ARF. Second, other techniques used two different transducers for generating multi-frequency excitation pulse and tracking induced motion “off-axis” to ARF whereas the proposed work uses a single transducer to perform both generation and tracking of motion. Third, the feasibility of generating multi-excitation motion in the homogeneous material has used only, whereas this work has shown the feasibility in 16 different inclusions with varying stiffnesses and sizes and tumors in a mouse model, in vivo.

The proposed continuous excitation pulse was generated by weighting sinusoids with different frequency components differently (j² in (1)), and the discrete excitation pulses were generated by sampling the continuous excitation pulse. Therefore, the energy content of each frequency can be different. This is evident in FIG. 3D, which shows that the Fourier transform of the differential displacements contains 10 distinct peaks at 100:100:1000 Hz with varying amplitude. Ten distinct peaks correspond to the frequency in the excitation pulse. Though the energy content of each frequency was different, the same excitation pulse was used to interrogate both background and inclusion. Therefore, the ratio of displacements of background over inclusion at each frequency reflects the ratio of stiffness of background over inclusion at the corresponding frequency. Note, ST-HMI generates an image of the relative stiffness of inclusion with respect to the background. Note, there was not much difference in contrast or CNR of ST-HMI-derived images due to the difference in energy content of 420 Hz.

While the displacements at frequencies corresponding to the frequencies of the excitation pulse were calculated, the differential displacement profiles also contained higher harmonic frequencies (i.e., greater than 1000 Hz). Displacement images at higher harmonic frequencies were not exploited. Because the energy of the frequency greater than the frequency of the excitation pulse is less controllable and depends on the relative location of the pixel, the harmonic generation can be different at the center of the inclusion versus near inclusion boundaries. At each pixel, the differential displacement profile was bandpass filtered to estimate P2PD at 100:100:1000 Hz by adaptively finding the cutoff values. As the cutoff values were found adaptively at each pixel, the cutoff values varied based on the pixel location.

The importance of generating P2PD at different frequencies to delineate different sized 36 kPa inclusions is qualitatively demonstrated in FIG. 4. Qualitatively, ARFI and P2PD images (frequency ≥300 Hz) contrasted 10.4 and 6.5 mm inclusions. However, 2.5 and 1.6 mm inclusions were not contrasted by ARFI images, whereas P2PD images at 900 and 1000 Hz were able to contrast 2.5 and 1.6 mm inclusions.

The further importance of exploitation of frequency for delineating inclusions with different sizes and stiffnesses is demonstrated quantitatively in FIGS. 5 and 6 in terms of contrast and CNR. The highest CNR and contrast were achieved at different frequencies depending on the inclusion size and stiffness. The maximum CNR and contrast achieved by ST-HMI were higher than ARFI irrespective of size and stiffness of inclusions. These results suggest the importance of using a multi-frequency excitation pulse to simultaneously generate displacement maps at different frequencies instead of using a pulsed excitation pulse to generate displacement profiles with a wide frequency range as it is done in ARFI.

The general trend in ST-HMI-derived CNR and contrast is that the frequency, at which maximum CNR and contrast were achieved, increases with increasing stiffnesses for fixed-size inclusion and increases with decreasing size for fixed stiffness inclusion. This is expected. Because, in a material with fixed stiffness, the wavelength of the generated shear waves within the ARF excitation beam can be smaller for higher frequency. Therefore, higher frequencies are better to contrast smaller inclusions. Similarly, the wavelength can be larger for the stiffer materials (i.e., higher shear wave speed) for a fixed frequency. Therefore, a higher frequency is needed to generate more than one wavelength of shear waves in a stiffer inclusion for the better detection of the inclusion. Note, the ST-HMI interrogates mechanical properties at the ARF-ROE without observing shear wave propagation away from the ARF-ROE. Therefore, the frequency is exploited to better detect inclusion due to the shearing within the ARF excitation beam.

The CNR and contrast mainly increased with frequency until its reached maximum and then decreased with frequency for 6 and 9 kPa inclusion irrespective of size. However, the CNR and contrast increased with frequency for 36 and 70 kPa inclusions with 2.5 and 1.6 mm diameters which suggests the further optimization in ST-HMI performance is possible by using a higher frequency for these inclusions.

The perceived size of the inclusions in the P2PD images depends on the frequency. As the CNR was calculated by using inclusion's and background's ROI of concentric circle and ring (FIG. 3A), the difference in estimated versus true size is reflected in the CNR values. Therefore, the frequency, where maximum CNR was achieved, can be used to reflect the true size of the inclusions.

While delineating the true boundary of lesions is useful in surgical planning or guiding biopsy or monitoring the response of the treatment, like, shrinkage of tumors due to the chemotherapy response), the P2PD ratio of background over inclusion can be used as a relative stiffness indicator for longitudinal or cross-sectional studies. FIG. 7 shows that the P2PD ratio is highly correlated with Young's moduli irrespective of frequencies or inclusion sizes. Therefore, the P2PD ratio at each frequency can be used to monitor the progression or regression of diseases.

These results in the phantoms are very promising. However, phantoms are the idealistic representation of tissues. In vivo performance of ST-HMI was evaluated by imaging tumors in a mouse model after 3 days of tumor cell implantation. Similar to the phantom, the P2PD ratio of healthy tissue to tumor varied with frequencies, and maximum (CNR, P2PD ratio) was achieved at (900, 900) and (900, 800) Hz for mouse #1 and #2, respectively. While both mice were imaged at 3-days after tumor cell implantation, the P2PD ratio was slightly different between mouse #1 versus 2. It can be due to the difference in response between mice after tumor cell implantation.

The feasibility of generating ST-HMI-derived P2PD at multi-frequency was presented using an excitation pulse composed of a sum of sinusoids with 100:100:1000 Hz. The performance of the proposed excitation pulse was evaluated by imaging 16 different inclusions with varying stiffnesses and sizes and was compared to the ARFI imaging. The highest CNR and contrast were achieved at a frequency dependent on the inclusion size and stiffness. The maximum CNR and contrast achieved by ST-HMI were higher than ARFI irrespective of inclusion size and stiffness. The P2PD ratio is highly correlated with Young's moduli irrespective of frequencies or sizes. Similar to the phantom, the P2PD ratio of healthy tissue over tumors varied with frequency in the animal. ST-HMI was capable of detecting as small as 1.6 mm diameter inclusion in phantom and 2.2 mm tumor in a mouse. These findings indicate the advantages of using a multi-frequency excitation pulse to simultaneously generate oscillation at several frequencies to better delineate inclusions or lesions.

### Example 2: Feasibility of Harmonic Motion Imaging Using Single Transducer: Experimental Demonstration in Breast Cancer Mouse Model and Breast Cancer Patients

Single Transducer Harmonic Motion Imaging (ST-HMI) Excitation and Tracking Pulse Sequence: In certain ST-HMI methods, AM-ARF can be generated by sinusoidally varying excitation pulse duration and collecting tracking pulses to estimate the excitation pulse induced displacements. Tracking pulses can be similar to 2-cycle B-mode imaging pulses, whereas excitation pulses can be long-duration pulses. Note, tissue displacement can linearly increase with execution pulse duration for fixed acoustic pressure. So sinusoidal variation in excitation pulse duration generates sinusoidally modulated displacements. Sinusoidal variation in excitation duration was generated using the following equation:

ep(t)=t_{ARF}^{min}+(t_{ARF}^{max}−t_{ARF}^{min})*sin(2πf_{HMI}t) (5)

where t is time, ep (t) overall continuous excitation duration, t_{ARF}^{min} and t_{ARF}^{max} are the minimum and maximum ARF exaction pulse duration, and f_{HMI} is the ST-HMI oscillation frequency. Equation (1) is the continuous form of the excitation pulse duration equation. However, integers number of excitation pulses can be used to accommodate tracking pulses by sampling the equation (5).

EP[n]=ep(t)*S(t−n(T−t_{offset})),n=1 . . . Nₑₚ (6)

where EP[n] is the discrete time excitation duration, T is HMI oscillation period (1/f_{HMI}), Nₑₚ is the maximum number of excitation pulses in a period, δ is the delta-dirac function, and t_{offset} is the excitation pulse offset to define the 1^{st} and last execution pulse time point in a period. As tracking pulses can be interleaved between excitation pulses, the number of tracking pulses can depend on Nₑₚ and tracking pulse repetition frequency (PRF). In certain ST-HMI embodiments, a reference tracking pulse can be sent first and displacement estimated with respect to the reference tracking pulse. An excitation pulse can be sent just after the reference tracking pulse if t_{offeset}=0 ms. However, tracking pulses can be sent until t_{offeset} if t_{offeset}>0 ms.

Measurements of Acoustic Field and Temperature Associated With ST-HMI Pulse Sequence: To evaluate the safety associated with the proposed ST-HMI pulse sequence, a variety of characteristics, including but not limited to, acoustic pressure and intensity of excitation pulses and temperature rise during the entire ST-HMI sequence, can be measured. In certain embodiments, acoustic pressure was measured by a calibrated broadband hydrophone (Model HGL-0020, Onda Corporation, Sunnyvale, Calif., USA) mounted on a mechanical stage and controlled by stepper motors. The experiment was performed by submerging the transducer and hydrophone in a water tank. The oscillation frequency, excitation pulse per cycle, focal depth, excitation pulse center frequency, and excitation voltage were fixed to 220 Hz, 8, 20 mm, 4.0 MHz, and 35 V, respectively. The derated mechanical index (MI_{0.3}) and derated spatial peak temporal average (I_{SPTA, 0.3}) can be calculated by derating measured pressure at a rate of 0.3/dB/cm/MHz. As the ST-HMI excitation can contain several pulses, the combined excitation I_{SPTA,0.3} can be calculated by summing the contribution of all pulses as I_{SPTA,0.3}=Σᵢ₌₁^{N}^{ₑₚ}PII_{0.3}ⁱ*f_{HMI} where PII_{0.3} is the derated pulse intensity integral. All hydrophone signals were digitized with a Tetronix oscilloscope (Tektronix, Inc, Beaverton, Oreg., USA).

In certain embodiments, temperature rise ST-HMI sequence can be measured by sandwiching a wire type thermocouple (Thermo Works IT-1K, UT, USA) between the transducer and a piece of dog's liver. Experiments can be performed at 37° C. water by positioning the thermocouple at the center of the ST-HMI lateral field of view. Thermocouple was posited first at 1 mm and then, 20 mm from the transducer surface to measure temperature rise at transducer surface and at focal depth, respectively. Two repeated measurements were taken at each position.

Phantom Assessments: In certain embodiments, imaging of two commercially available phantoms (customized model 049A, CIRS, Norfolk, VA) was performed using a Verasonics research scanner (Vantage 256, Verasonics Inc., Kirkland, Wash., USA) with L7-4 (Philips Healthcare, Andover, Mass., USA) transducer. In both phantoms, three stepped cylinder inclusions with varying diameters and stiffness were embedded in the same stiffness background. The manufacturer provided Young's modulus of 6 inclusions and background was 8±1.5, 10±2, 15±3, 20±4, 40±8, 60±10, and 5±1.0 kPa, respectively. In all inclusions, ST-HMI can be performed using f_{HMI}, Nₑₚ, t_{offset}, and oscillation cycle number of 220 Hz, 8, 0.2 ms, and 5, respectively. For two-dimensional imaging, thirty four evenly spaced A-lines were acquired across 20 mm lateral field of view (FOV). Preceding each 2-D ST-HMI acquisition was one spatially-matched B-mode image, with 128 lateral lines spanning a lateral FOV of approximately 38 mm, for anatomical reference.

Mechanical properties of biological tissues can depend on their underlying microscopic and macroscopic structures and compositions. So, the changes in mechanical properties can be associated with a broad spectrum of pathologies, given that diseases change the structures and compositions of molecular building blocks of tissues. Mechanical properties of tissues can be used or assessed either using ultrasound elastography (UE) or magnetic resonance elastography (MRE), or optical coherence elastography (OCE). UE can be favorable in certain cases due to its low cost, ease of use, portability, ability to penetrate deeper in tissue, and ability to characterize the motion within the human body. Over the last three decades, certain UE methods for interrogating mechanical properties have been developed and can be applied to diagnose diseases in the liver, breast, thyroid, prostate, kidney, muscles, carotid artery, and lymph nodes.

Among certain UE approaches are those that exploit acoustic radiation force (ARF) to induce motion within a tissue. ARF-based methods either use displacements “on-axis” to the ARF or shear wave propagation “off-axis” to the ARF or both to assess mechanical properties of tissues. Both “on-axis” and “off-axis”-based methods can have their own pros and cons. Generally, “off-axis” shear wave based methods can provide quantitative mechanical properties like stiffness and viscosity. However, shear wave based measurements can be subject to artifacts from shear wave reflections and distortions in the finite, heterogeneous media. Moreover, shear wave measurement is generally performed over a 2-5 mm lateral window, leading to reductions in spatial resolution of mechanical property. Finally, as the displacement can reduce “off-axis” to ARF with shear wave propagation due to diffraction and dispersion, applications in deeper organs, obese patients, and stiffer tissues can be limited.

In contrast to certain “off-axis” measurements, certain “on-axis” displacements can provide a qualitative assessment of mechanical properties as the force or stress are generally unknown. However, there can be several benefits to the “on-axis” evaluation of displacements. First, interference from certain reflected or distorted shear waves can be less likely as the displacements are observed immediately following the ARF excitation. Second, displacements can be measured pixel-by-pixel basis without lateral averaging, so the finer spatial resolution of mechanical features is supported. Third, displacements can be greatest at on-axis to ARF excitation, so “on-axis” methods can assess mechanical properties in deeper organs, obese patients, and stiffer tissues.

Methods based on the observation of “on-axis” ARF response include acoustic radiation force impulse (ARFI) imaging, kinetic acoustic vitreoretinal examination (KAVE), monitored steady-state excitation recovery (MSSER), ARF creep imaging, viscoelastic response (VisR) ultrasound imaging, and harmonic motion imaging (HMI). A difference between certain HMI methods with other “on-axis” based methods is that amplitude modulated-ARF (AM-ARF) can be used to generate harmonic oscillation of tissue, whereas transient ARF is used in other methods. The advantage of using harmonic excitation is the fact that motion at the input oscillation frequency can be easily filtered from reverberation, movement, and breathing artifacts. Previously, certain HMI methods have been used for detecting pancreatic tumors, monitoring treatment response of pancreatic tumor, monitoring of high intensity focused ultrasound (HIFU) ablation of the tumor, and livers. In certain HMI setups, a focused ultrasound (FUS) and an imaging transducer can simultaneously generate and track AM-ARF-induced motion. Certain uses of two different transducers in HMI to induce and estimate harmonic motion renders the HMI system highly complex to use for diagnostic imaging. The data acquisition can be facilitated if the generation and tracking of harmonic motion could be performed by a single imaging transducer.

Towards a goal of facilitating certain HMI data acquisitions, the disclosed subject matter shows the feasibility of generating and mapping harmonic motion using a linear array transducer. This HMI method, named single transducer-HMI (ST-HMI), can generate AM-ARF by varying the excitation pulse duration and estimate motion by collecting tracking pulses in between the excitation pulses. Note, the change in excitation pulse duration can change the Iₛₚₜₐ of the pulse, which can generate different magnitude ARF. Previously, certain methods generated and tracked harmonic waves using a single transducer in the shear wave dispersion ultrasound vibrometry (SDUV) method to assess the viscoelastic properties of certain tissues. However, a pulsed ARF excitation can oscillate at a particular frequency in SDUV rather than modulating the excitation pulse duration. Due to the oscillation of ARF excitation pulse, SDUV produces shear waves with multiple harmonics with comparable amplitude to the fundamental frequency, and the wave energy can be distributed over several harmonic, which can limit its application in certain low SNR scenarios. Certain embodiments have developed harmonics shear wave imaging (HSWI) to generate narrowband shear waves by modulating ARF excitation pulse duration and showed an amplitude of fundamental frequency several times higher than the harmonics frequencies. However, HSWI can be an “off-axis” ARF-based method, and the performance of HSWI was validated in homogeneous materials.

The feasibility of generating and tracking harmonic motion using a single transducer is demonstrated in contrasting inclusions with varying degrees of stiffness. ST-HMI-derived displacements can be used to contrast inclusions from the background. The impact of oscillation frequency, oscillation cycle, and ARF excision pulses per cycle in contrasting inclusions is evaluated. Third, the feasibility of longitudinal monitoring of tumor progression in breast cancer mouse models using ST-HMI with a high-frequency transducer is tested. Fourth, the feasibility of contrasting tumors in breast cancer patients is demonstrated.

Harmonic motion imaging (HMI) can interrogate the mechanical properties of tissues by simultaneously generating and tracking amplitude-modulated (AM) force-induced motion using a focused ultrasound and an imaging transducer, respectively. Certain advantages of HMI include that motion at oscillation frequency can be easily filtered from different motion artifacts, and viscosity can be estimated using motion at variable frequencies. However, the use of two separate transducers can render the HMI system highly complex to use for diagnostic imaging. The objective is to develop an ST-HMI technique for generating and mapping HM using a linear array transducer to facilitate data acquisition.

In certain ST-HMI embodiments, the AM force can be generated by varying the duration of HMI excitation pulses, and motion can be estimated by acquiring tracking pulses interleaved with excitation pulses. A Verasonics Vantage system and L7-4 (phantom and Human) or L22-14vxLF (Mouse) arrays with (excitation, tracking) pulses' center frequency of (4.0, 6.1) or (15.63, 20.8) MHz, respectively, can be used. ST-HMI was tested in phantoms with 8±1.5, 10±2, 15±3, 20±4, 40±8, and 60±10 kPa inclusions embedded in 5±1 kPa background (CIRS, Norfolk), 4T1 breast tumors in mice in vivo (N=4), and breast cancer patients in vivo (N=2; 53F and 23F with invasive ductal carcinoma (IDC) and fibroadenoma (FA), respectively). In certain embodiments, the 5-cycle oscillation of 220 Hz was used except in 60 kPa inclusions where oscillation was varied from 60 to 420 Hz to find the effect of frequency on ST-HMI rendered image.

In phantom, the mean background-to-inclusion peak-to-peak displacement (P2PD) ratio was well correlated with Young's moduli ratio (R2=0.93). CNR and contrast in the 60 kPa inclusion were highest in the order of 180-Hz frequency, which indicated that the ST-HMI frequency can be tailored to the stiffness for best performance. In mice, the P2PD ratio of healthy-to-tumor tissue increased over 4 weeks of post tumor cell injection, indicating stiffening, and ST-HMI could track the changes. In patients, the healthy-to-tumor P2PD ratio was 4.35 and 1.51 in IDC and FA, respectively. The disclosed subject matter demonstrates that ST-HMI can be used for tracking changes in the viscoelasticity of cancerous tissues during disease progression and treatment.

Harmonic motion imaging (HMI) interrogates the mechanical properties of tissues by simultaneously generating and tracking amplitude-modulated acoustic radiation force (AM-ARF)-induced motion using a focused ultrasound and an imaging transducer, respectively. Instead of using two transducers, the disclosed subject matter provides a technique directed to single transducer HMI (ST-HMI), a novel method to assess mechanical properties, to facilitate data acquisition. In ST-HMI, AM-ARF was generated by modulating excitation pulse duration, and tracking of harmonic motion was performed by collecting tracking pulses in between the excitation pulses. The feasibility of ST-HMI was performed by imaging commercially available two elastic phantoms with three inclusions (N=6), in vivo monitoring of changes in the tumor's stiffness of 4T1, orthotropic breast cancer mouse model (N=4), and patients (N=3) with breast masses in vivo. Six inclusions with nominal Young's modulus of 8, 10, 15, 20, 40, and 60 kPa embedded in 5 kPa background. ST-HMI-derived peak-to-peak displacements (P2PD) successfully contrasted all 6 inclusions, and the P2PD ratio of background over inclusion was highly correlated with Young's moduli ratio of inclusion over the background with R² of 0.93. The median P2PD ratio of the tumor over healthy tissue was 3.0, 5.1, 6.1, and 7.7 at 1, 2, 3, and 4 weeks post-injection of the tumor cell. ST-HMI detected breast masses with fibroadenoma (FA), pseudo angiomatous stromal hyperplasia (PASH), and invasive ductal carcinoma (IDC) with the P2PD ratio of 1.37, 1.61, and 1.78, respectively. These results suggest that ST-HMI assesses the mechanical properties of tissue via the generation and tracking of harmonic motion in the region of ARF excitation.

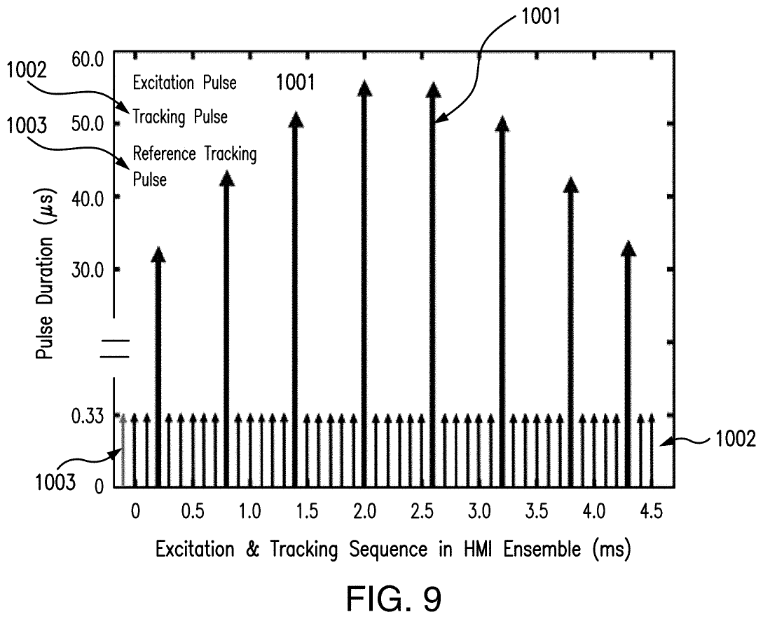

Single Transducer Harmonic Motion Imaging Excitation and Tracking Pulse Sequence: In ST-HMI, AM-ARF was generated by sinusoidally varying excitation pulse duration, and tracking pulses were collected in between excitation pulses to estimate the induced displacements (FIG. 9). Tracking pulses were like typical 2-cycle B-mode imaging pulse, whereas excitation pulses are long duration pulse. Note, tissue displacement linearly increases with excitation pulse duration for fixed acoustic pressure. So sinusoidal variation in excitation pulse duration generates sinusoidally modulated displacements. Sinusoidal variation in excitation duration (ed (t)) was generated using the equation (5).

Equation (5) is the continuous form of the excitation pulse duration. However, integers number of excitation pulses were used to accommodate tracking pulses by sampling the equation (5) as shown in equation (6).

As tracking pulses were interleaved between excitation pulses, the number of tracking pulses depends on Nₑₚ and tracking pulse repetition frequency (PRF). In ST-HMI, a reference tracking pulse was transmitted first, and displacement was estimated with respect to the reference tracking pulse. An excitation pulse was transmitted just after the reference tracking pulse if t_{offset}=0 ms. However, tracking pulses were collected until t_{offset} if t_{offset}>0 ms (FIG. 9).

Safety Measurements Associated with Single Transducer Harmonic Motion Imaging Pulse Sequence: To evaluate the safety associated with the proposed ST-HMI pulse sequence, acoustic pressure and intensity of excitation pulses, and temperature rise during the entire ST-HMI sequence was measured. Acoustic pressure was measured by a calibrated broadband hydrophone (Model HGL-0020, Ondo Corporation, Sunnyvale, Calif., USA) mounted on a mechanical stage and controlled by stepper motors. The experiment was performed by submerging the hydrophone and L7-4 transducer (Philips Healthcare, Andover, Mass., USA) in a water tank. The oscillation frequency, excitation pulse per cycle, focal depth, excitation pulse center frequency, and excitation voltage were fixed to 220 Hz, 8, 20 mm, 4.0 MHz, and 35 V, respectively. The derated mechanical index (MI_{0.3}), spatial peak temporal average (I_{SPTA,0.3}), and spatial peak pulsed average (I_{SPPA,0.3}) were calculated by derating measured pressure at a rate of 0.3/dB/cm/MHz. As the ST-HMI excitation contains several pulses, the combined excitation I_{SPTA,0.3} was calculated by summing the contribution of all pulses as I_{SPTA,0.3}=Σᵢ₌₁^{N}^{ₑₚ} PII_{0.3}ⁱ*f_{HMI} where PII_{0.3} is the derated pulse intensity integral. I_{SPPA,0.3} was calculated as

$${I}_{SPTA , 0.3} = \frac{{\sum }_{i = 1}^{{N}_{e p}} {PII}_{0.3}^{i}}{{\sum }_{t = 1}^{{N}_{e p}} P {D}^{i}}$$

where PDⁱ is the duration of i^{th} excitation pulse. All hydrophone signals were digitized with a Tetronix oscilloscope (Tektronix, Inc, Beaverton, Oreg., USA).

Temperature rise due to the entire ST-HMI sequence was measured by sandwiching a needle-type thermocouple (Thermo Works T-29X, UT, USA) between the transducer and a piece of dog's liver. The experiment was performed at 37° C. water by positioning the thermocouple at the center of the ST-HMI lateral field of view (FOV). The thermocouple was posited first at 1 mm and then, 20 mm from the transducer surface to measure temperature rise at transducer surface and focal depth, respectively. Two repeated measurements were taken at each position.

Phantom Assessments: Imaging of two commercially available phantoms (customized model 049A, CIRS, Norfolk, VA) was performed using a Verasonics research scanner (Vantage 256, Verasonics Inc., Kirkland, Wash., USA) with an L7-4 transducer. In both phantoms, three stepped-cylindrical inclusions with varying diameters and stiffness were embedded in a 5±1.0 kPa background. The manufacturer provided Young's modulus of 6 inclusions was 8±1.5, 10±2, 15±3, 20±4, 40±8, and 60±10. Imaging was performed at 10±1.0 mm cross-section of the cylindrical inclusions. In all inclusions, ST-HMI was performed using f_{HMI}, Nₑₚ, t_{offset}, and oscillation cycle numbers of 220 Hz, 8, 0.2 ms, and 5, respectively, with the parameters indicated in Table 2. For two-dimensional imaging, thirty-four evenly spaced A-lines were acquired across approximately 20 mm lateral FOV. Preceding each 2-D ST-HMI acquisition was one spatially-matched B-mode image, with 128 lateral lines spanning a lateral FOV of approximately 38 mm, for anatomical reference.

[symbol]

TABLE 2
Excitation and tracking parameters of single transducer
harmonic motion imaging used in phantoms, in
vivo breast cancer patients, and mice.

| | | |
|---|---|---|
| | Phantom/ | |
| Parameters | Human | Mouse |

Beam sequence parameters of ST-HMI/ARFI

| Transducer | L7-4 | | 1.22- | |
|---|---|---|---|---|
| | | | 14vXLF | |
| Bandwidth | 58% | | 51% | |
| Sampling frequency | 20.84 | MHz | 62.5 | MHz |
| Acoustic lens axial focus | 25 | mm | 20 | mm |
| Excitation pulse frequency | 4.0 | MHz | 15.6 | MHz |
| Excitation pulse F-number | 2.25 | | 2.25 | |
| Tracking pulse frequency | 6.1 | MHz | 20.8 | MHz |
| Tracking pulse transmit F-number | 1.75 | | 1.75 | |
| Tracking pulse receive F-number* | 1.0 | | 1.0 | |
| Excitation and tracking | 15 mm (Pha.) | | 11.3 + | |
| pulse axial focus | 14 + 3.6 mm | | 0.5 mm | |
| | (Human) | | | |
| Minimum excitation pulse | 28 | μs | 20 | μs |
| duration (t [symbol] , ST-HMI) | | | | |
| Maximum excitation | 55 | μs | 30 | μs |
| pulse duration (t [symbol] , ST- | | | | |
| HMI) | | | | |
| Oscillation frequency | 60-420 Hz | | 200 | Hz |
| (ST-HMI) | (Phantom) | | | |
| | 220 Hz | | | |
| | (Human) | | | |
| Oscillation cycle number | 2-10 | | 5 | |
| (ST-HMI) | | | | |
| Single excitation pulse | 87.5 | μs | — | |
| duration (ARFI) | | | | |
| Tracking pulse number | 110 | | — | |
| (ARFI) | | | | |
| Tracking pulse PRF | 10 | KHz | 10 | KHz |
| Spacing between RF-lines | 0.59 | mm | 0.3 | mm |
| RF-lines number per 2-D image | 34 | | 14 | |
| Lateral field of view size | 20 | mm | 4.2 | mm |

Normalized cross correlation parameter

| Interpolation factor | 4 | | 4 | |
|---|---|---|---|---|
| Kernel length | 592 | μm | 295 | μm |
| Search region | 80 | μm | 80 | μm |

*Aperture growth and dynamic Rx focusing enabled [symbol]
indicates data missing or illegible when filed

Besides performing ST-HMI imaging in different stiffness inclusions, the impact of f_{HMI}, Nₑₚ, t_{offset}, and oscillation cycle number on ST-HMI images were evaluated. The impact of oscillation frequency was investigated by imaging 15±3 kPa and 60±10 kPa inclusions with varying f_{HMI} from 60 Hz to 420 Hz in steps of 40 Hz. Excitation pulses per cycle were varied between oscillation frequencies to keep the I_{SPTA} (i.e., the duty cycle of excitation pulse) constant. The duty cycle of the excitation pulse was calculated as 100*f_{HMI}*Σᵢ₌₁^{N}^{ₑₚ} t_{ARF}ⁱ where t_{ARF}ⁱ was the duration of the i^{th} excitation pulse. Excitation pulse duty cycle was kept around 8% by using Nₑₚ of 30, 18, 13, 10, 8, 7, 6, 5, 5, and 4 for f_{HMI} of 60, 100, 140, 180, 220, 260, 300, 340, 380, and 420 Hz, respectively. Oscillation cycle number and t_{offset} were fixed to 5 and 0.2 ms, respectively, for all oscillation frequencies.

To investigate the effect of duty cycle on ST-HMI's performance, the same two inclusions were imaged with variable (duty cycle, Nₑₚ) of (3.8%, 5), (6.36%, 8), (8.88%, 11), and (11.36%, 14) and with fixed f_{HMI}, t_{offset}, and oscillation cycle number of 180 Hz, 0.2 ms, and 5, respectively.

The oscillation cycle number was varied from 2 to 10 in steps of 2 with fixed f_{HMI}, Nₑₚ, and t_{offset} of 420 Hz, 4, and 0.2 ms, respectively. Finally, the effect of the t_{offset} was evaluated by varying from 0 to 0.6 ms in steps of 0.2 ms with fixed f_{HMI}, Nₑₚ, and oscillation cycle numbers of 180 Hz, 10, and 5, respectively. It is not possible to change the t_{offset} without changing the duty cycle of the excitation pulse. There was a slight change in the duty (7.4-8.5%) for the t_{offset} of 0-0.6 ms.

For each case, 6 repeated acquisitions were performed by moving the transducer in the elevational direction. Each inclusion can be represented by its mean nominal Young's moduli values.

In Vivo Breast Cancer Mouse Imaging: Orthotropic, 4T1 breast cancer mice (N=4) were used to investigate the performance of ST-HMI in monitoring longitudinal change in tumor stiffness. The induction of cancer and imaging protocols was reviewed and approved by the Institutional Animal Care and Use Committee (IACUC). 8-10-weeks-old female BALB/c mice were purchased from the Jackson Laboratory. Cancer was inducted by injecting 2×10⁵ 4T1 breast cancer cells in the 4^{th} inguinal mammary fat pad.

ST-HMI was performed using a Verasonics research scanner with L22-14vXLF (Vermon, Tours, France) transducer. Mice were anesthetized with 1-2% isoflurane in oxygen throughout the imaging procedures. Imaging was performed by placing mice in a supine position on a heating pad with their abdominal hair removed. The transducer was placed in a container filled with degassed water and an acoustically transparent membrane at the center. The tumor was positioned underneath the membrane with degassed ultrasound gel in between the tumor and membrane.

Mice were imaged at 1, 2, 3, and 4 weeks post-injection of tumor cell using the parameters indicated in Table 1. f_{HMI}, Nₑₚ, t_{offset}, and oscillation cycle numbers were fixed to 200 Hz, 13, 0.7 ms, and 5, respectively. 2-D HMI image was formed by acquiring fourteen evenly spaced A-lines across 4 mm lateral FOV. One spatially matched B-mode image was acquired with 128 lateral lines spanning a lateral FOV of approximately 13.6 mm, for anatomical reference. Tumors grew larger over time. If the tumor's size was higher than the ST-HMI lateral FOV, multiple acquisitions were acquired by mechanically translating the transducer in lateral directing using a 3-D positioning system (Velmex Inc., Bloomfield, NY, USA). The Final image was formed by stitching all acquisitions.

Imaging of Patients with Breast Masses

Clinical performance of ST-HMI was evaluated by imaging female patients with breast masses (N=3) following human subjects protocol approval by the CUIMC Institutional Review Board (IRB). Informed consent was obtained from all enrolled subjects. Two patients with suspicious breast masses were scheduled to undergo needle biopsy, and one patient with invasive ductal carcinoma (IDC) was scheduled for the breast segmentectomy. Similar to the phantom experiments, ST-HMI imaging was performed using a Verasonics research scanner with L7-4 with parameters in table 1. Biopsies patients were asked to remain motionless during imaging. The patient with the malignant tumor was imaged after general anesthesia, intraoperatively. Patients were imaged in a supine or lateral oblique position, and the location and boundaries of tumors were confirmed by an experienced sonographer in the B-mode ultrasound image. Data were collected by orienting the transducer parallel to the radial direction (i.e., line connecting center of mass and nipple). Imaging focal depth was set at the center of the breast mass.

Single Transducer Harmonic Motion Imaging Data Processing: For all acquisitions, channel data were transferred to the computational workstation for offline processing using MATLAB (MathWorks Inc., Natick, Mass., USA). Custom delay and sum beamforming was applied to construct beamformed RF data. Motion tracking with respect to the reference tracking pulse was performed using one-dimensional normalized cross-correlation (NCC) with parameters as indicated in Table 1. After motion tracking, a 3-D dataset (axial×lateral×time) describing axial displacements overtime was generated. At each spatial location (axial×lateral), low-frequency motion due to the transient effect of the ARF was suppressed using differential displacements, which was computed by subtracting displacements between successive time samples. Then, the desired oscillation (f_{HMI}) was filtered out using a second-order Butterworth (butter and filter function) bandpass filter.

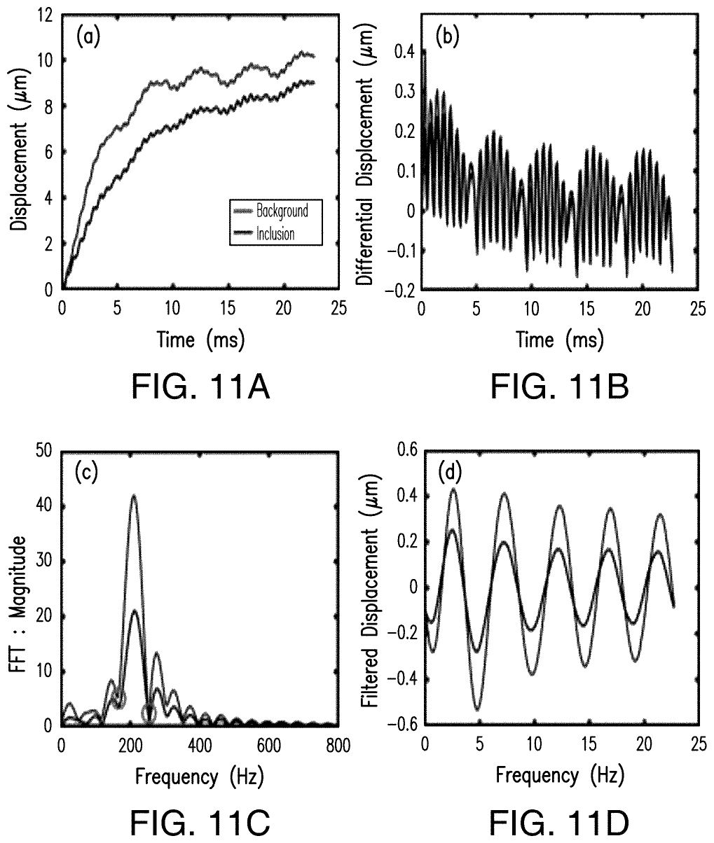

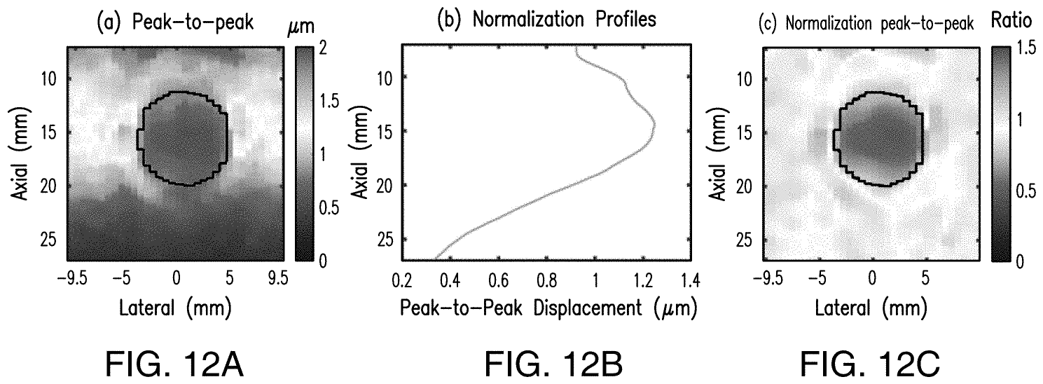

The cutoff values of the bandpass filter were selected adaptively for each data acquisition. The cutoff values were calculated by finding the frequency around f_{HMI} at which the 1^{st} minimum in Fast Fourier Transform (FFT) magnitude occurred (circle in FIG. 11C). Minimum FFT magnitude around f_{HMI} was found by calculating the successive difference in FFT magnitude and then finding the change in sign (sign function) in differential magnitude. As an example, the sign of differential magnitude was changed from negative to positive and positive to negative for lower and higher cutoff values. Adaptive cutoff values were calculated at selected locations instead of all pixels to expedite the data processing. The selected locations (axial, lateral) were ([focal depth and focal depth±5 mm], [−9.5, −4.5, 0.5, 5.5, and 9.5 mm]) and ([focal depth], [−2.0, −1.0, 0.0, 1.0, and 2.0 mm]) for imaging involving transducer L7-4 and L22-14vXLF, respectively. Then, the final lowest and highest cutoff values for filtering all pixels were the median of lower and higher cutoff values derived in the selected regions. After filtering out the desired oscillations, a 2-D image was formed by calculating average peak-to-peak displacement (P2PD) over cycles for each pixel (FIG. 12A).

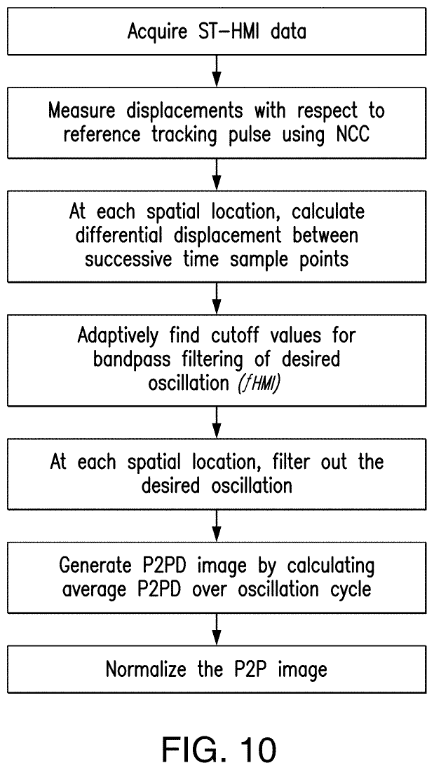

The P2PD is a function of ARF amplitude, and ARF amplitude varies over the axial range. So, the depth-dependent variation in P2PD can be normalized before P2PD can be compared over the axial range. The normalizing term [symbol] was derived as the median P2PD(x) over a lateral range spanning a presumed mechanically homogeneous region, i.e., for each axial location (x), median P2PD over a lateral range was computed. Then, the final normalized 2-D P2PD image was constructed by dividing each lateral line by the normalizing term [symbol] . The normalizing process assumes that ARF in the selected lateral range is similar to the ARF in all lateral locations. So the final normalized image represents the stiffness with respect to the stiffness of the selected homogenous region. Similar normalization was performed for ARF impulse (ARFI)-derived displacement image and Viscoelastic response derived relative elasticity and viscosity image. FIG. 10 depicts a flow chart representing the processing procedure implemented to generate normalized P2PD images.

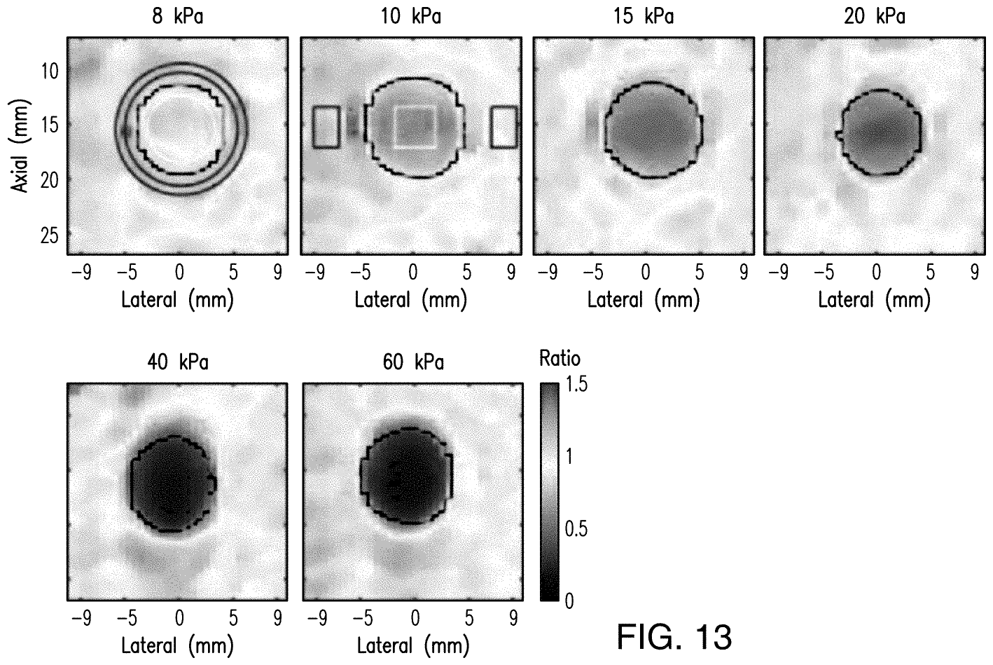

Image Quality Metrics: Contrast and contrast to noise ratio (CNR) were used to quantitatively evaluate the ST-HMI performance in inclusions. For contrast and CNR calculations, the region of interest (ROI) inside the inclusion was defined as the concentric circle with 80% of the corresponding inclusion's radius. Background ROI was a ring surrounding the corresponding inclusion, with an inner radius of 120% of the corresponding inclusion' radius. The outer radius was varied between inclusions depending on its size so that the area of inclusion and background's ROI was equal. See FIG. 13 for a depiction of inclusion (white circle) and background (black ring) ROIs. Contrast and CNR were calculated as:

$$Contrast = \frac{\vert {μ}_{INC} - {μ}_{B K D} \vert}{{μ}_{B K D}} \qquad ( 9 )$$

$$CNR = \frac{\vert {μ}_{INC} - {μ}_{B K D} \vert}{\sqrt{( {σ}_{INC}^{2} + {σ}_{B K D}^{2} )}} \qquad ( 10 )$$

where, μ and σ are the median and standard deviation of normalized P2PD in the inclusion's (INC) and background's (BKD) ROI.

The P2PD ratio of background over inclusion was compared with Young's moduli ratio of inclusion over a background by drawing rectangle ROI to avoid boundary impact. Inclusion rectangular ROI was defined as a rectangle with a height and width of 40% of inclusion's radius. Background ROI was defined as the two rectangles positioned 3.5 mm from the inclusion boundary, each with a height equal to the height of inclusion's ROI and width equal to the half of width of inclusion's ROI. See FIG. 13 for a depiction of rectangular ROI in inclusion and background. Inclusion boundary was derived from the B-mode ultrasound image.

Statistical Analysis

All statistical analyses were carried out using MATLAB. Ten separate Kruskal-Wallis testa (kruskalwallis function), were carried out to compare the contrast and CNR of ST-HMI derived images across different inclusions (8, 10, 15, 20, 40, and 60 kPa), across oscillation frequencies (60 to 420 Hz with steps of 40 Hz), across excitation pulse duty cycle (3.82, 6.36, 8.88, and 11.36%), across oscillation cycle number (2 to 10 with steps of 2), and across excitation pulse offset (0 to 0.6 ms with steps of 0.2 ms). If any group was statistically significant, two-sample Wilcoxon signed rank-sum test (signrank function) was used to find which combination was statistically significant. The P2PD ratio of background over inclusion was compared to Young's moduli ratio of inclusion over a background by calculating R2 of the linear regression between P2PD and Young's moduli ratio.

Two separate Kruskal-Wallis tests were carried out to compare mouse tumor diameters and P2PD ratios of healthy tissue over tumor across imaging time points (1, 2, 3, and 4 weeks post tumor cell injection). If any group was statistically significant, two-sample Wilcoxon rank-sum tests (ransum function) were used to find which combination was statistically significant. For all analyses, statistical significance was based on a two-sided α of 0.05.

FIG. 9 shows the excitation and tracking pulse sequence for one-period oscillation with frequency, excitation pulse per cycle, and excitation pulse offset of 220 Hz, 8, and 0.2 ms, respectively. The duration of excitation pulses 1001 was varied to generate AM-ARF, whereas the tracking pulse 1002 duration was fixed. This pulse sequence was repeated to generate 5 cycles of oscillation at each lateral line. A motion was estimated with respect to the reference pulse 1003. The MI_{0.3}, I_{SPTA,0.3} and I_{SPPA,0.3} was 1.37, 10.5 Wcm⁻², and 194.38 Wcm⁻². The median temperature rise due to the entire beam sequence was 0.4° C. and 0.6° C. at the focal depth (20 mm) and the surface of the transducer, respectively.

FIG. 11A shows two representative displacement profiles as a function of time in 5 kPa background and 15 kPa inclusion. Due to the initial transient effect, displacement increased slowly without observable oscillation (time <10 ms). 220 Hz oscillation was better observable when displacement reaches the steady-state. FIG. 11B shows the differential displacements of the same two profiles. The envelope of differential displacements clearly shows 220 Hz oscillation which was confirmed by the peak at 220 Hz in the fast Fourier transform (FFT) of differential displacement profiles (FIG. 11C). FIG. 11D represents the filtered displacement profiles. The average P2PD was 0.74 and 0.38 in the background and inclusion filtered displacement profiles, respectively.

A 2-D image was generated by calculating P2PD at each pixel (FIG. 12A). However, P2PD varies over axial range due to the variation in ARF amplitude over depth (FIGS. 12A-12B). Depth normalization profiles (FIG. 12B) was generated from the background to reconstruct normalized P2PD image (FIG. 12C).

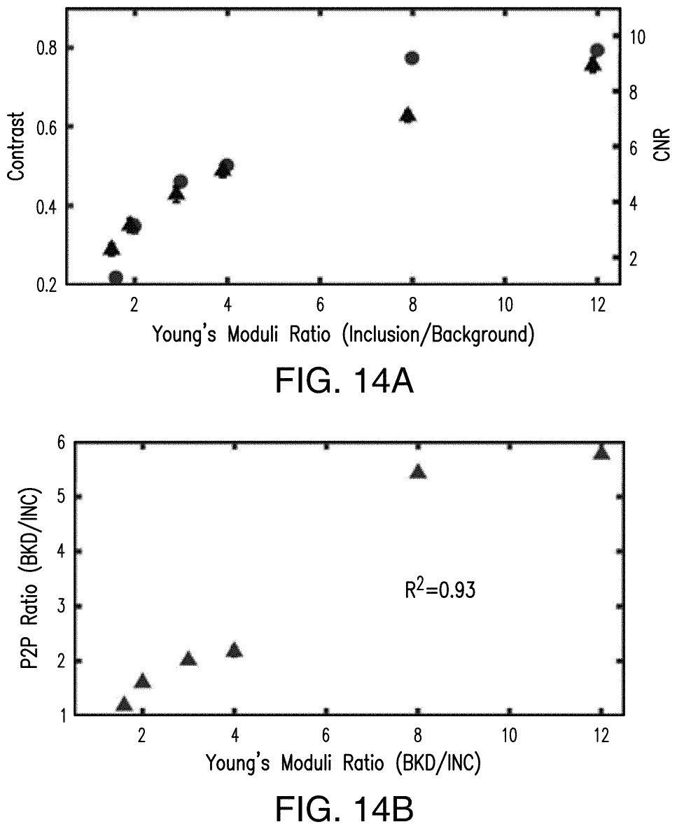

FIG. 13 qualitatively compares the normalized P2PD images of 8, 10, 14, 15, 20, 40, and 60 kPa inclusions embedded in 5 kPa background. The inclusion contrast increased with increasing Young's modulus of inclusion which is also evident in FIG. 14. FIG. 14A shows that contrast and CNR increase with increasing Young's moduli ratio of inclusion over the background. P2PD ratio of background over inclusion was highly correlated with Young's moduli ratio of inclusion over background (FIG. 14B). All inclusions were imaged with an oscillation frequency of 220 Hz.

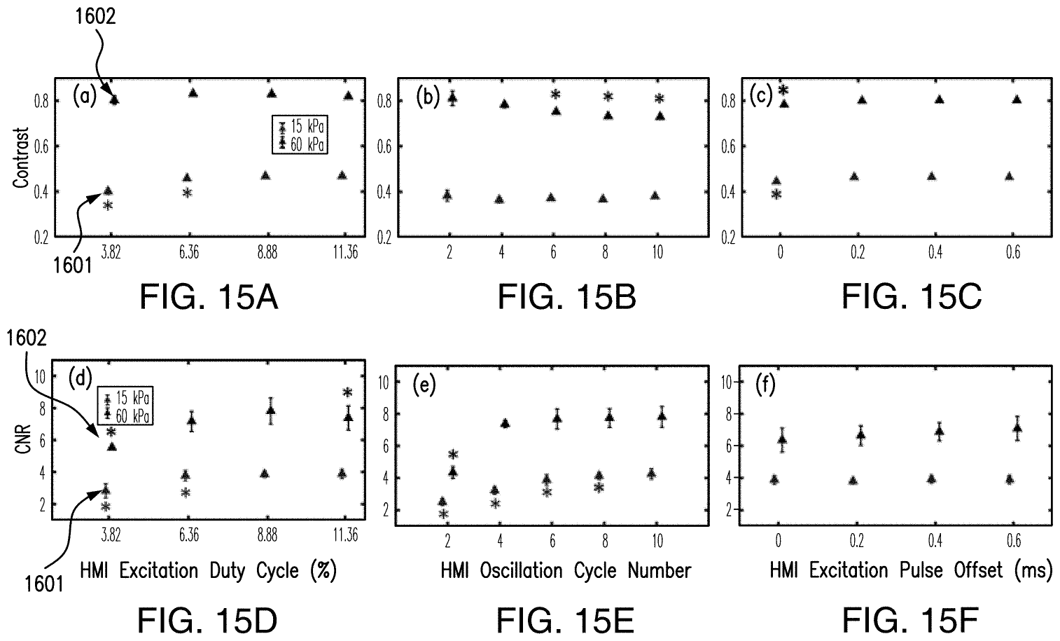

FIGS. 15A-15F qualitatively demonstrates the impact of oscillation frequency in contrasting 15 kPa 1601 and 60 kPa 1602 inclusions. Two observations are notable. First, the perceived size of the inclusion in the ST-HMI derived image became similar to the true size with higher frequencies for both phantoms. Second, lower frequencies (60 and 180 Hz) distorted the size more for 60 versus 15 kPa inclusion. FIG. 15 demonstrates that contrast and CNR of 15 and 60 kPa inclusions increases with frequency up to 140 and 220 Hz, respectively and then, reaches a plateau. Kruskal-Wallis test suggested that contrast and CNR were statistically different across frequencies for both inclusions. The highest median (contrast, CNR) was occurred at (180, 300) Hz and (180, 260) Hz for 15 and 60 kPa inclusions, respectively. The signed-rank sum test suggests that contrasts at 180 Hz were statistically different from all other frequencies for both inclusions, and CNR at 260 and 220 Hz was statistically different from all frequencies except 260 Hz and from frequencies less than 220 Hz for 15 and 60 kPa inclusions, respectively.

FIG. 15 shows the effect of excitation duty cycle (left column), oscillation cycle number (center column), and excitation pulse offset (right column) on the contrast (top row) and CNR (bottom row) of 15 and 60 kPa inclusions. Four observations are notable. First, the highest median contrast and CNR was achieved for duty cycle, cycle number, and offset of (6.36, 11.36) %, (2, 2), (0.4, 0.4) ms, and (8.88, 11.36) %, (10 10), (0.4, 0.6) ms for (15, 60) kPa inclusions respectively. Second, contrast and CNR did not change significantly after the duty cycle of 3.82% and 6.36% for 60 and 15 kPa inclusion, respectively. Third, the contrast was generally higher for lower oscillation cycle numbers, but CNR was higher for higher cycle numbers. Fourth, CNR was not impacted by the change in offset, and contrast did not change for offsets greater than 0 ms for both phantoms. The lowest contrast was achieved at t_{offset} of 0 ms, which also had the lowest duty cycle.

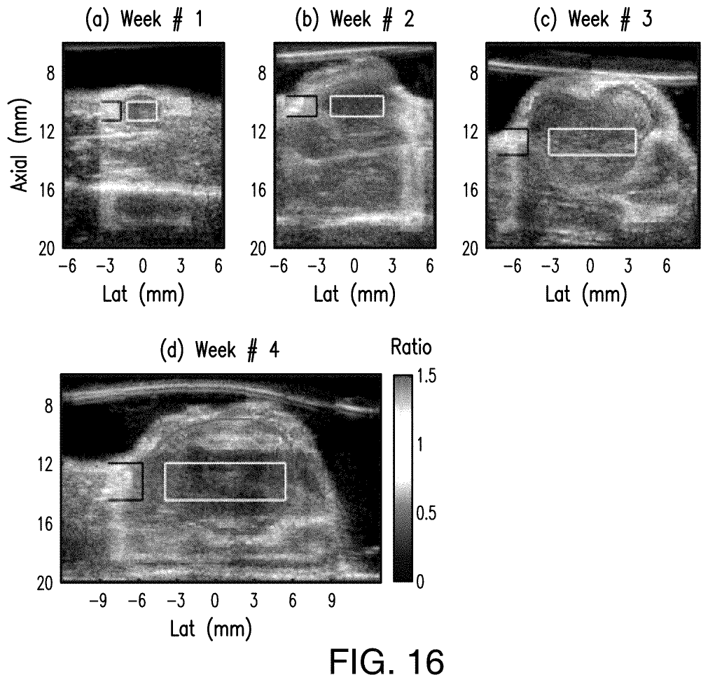

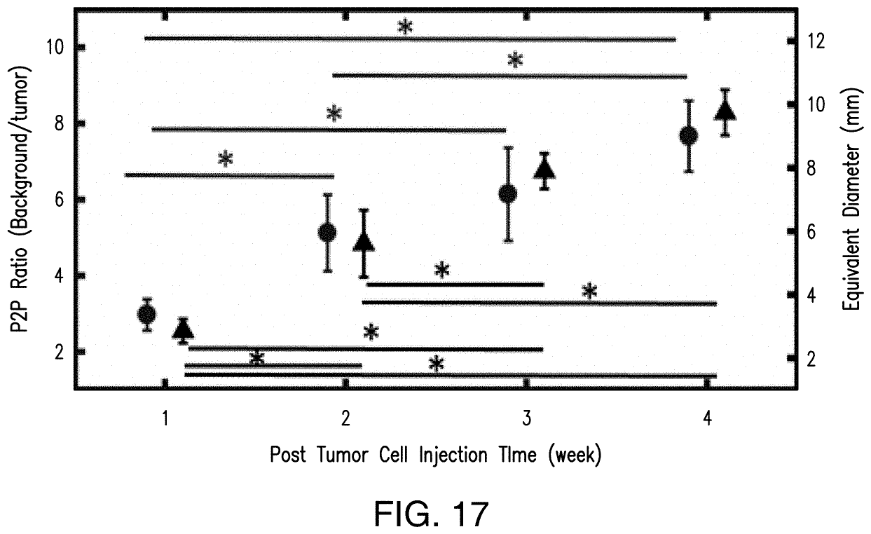

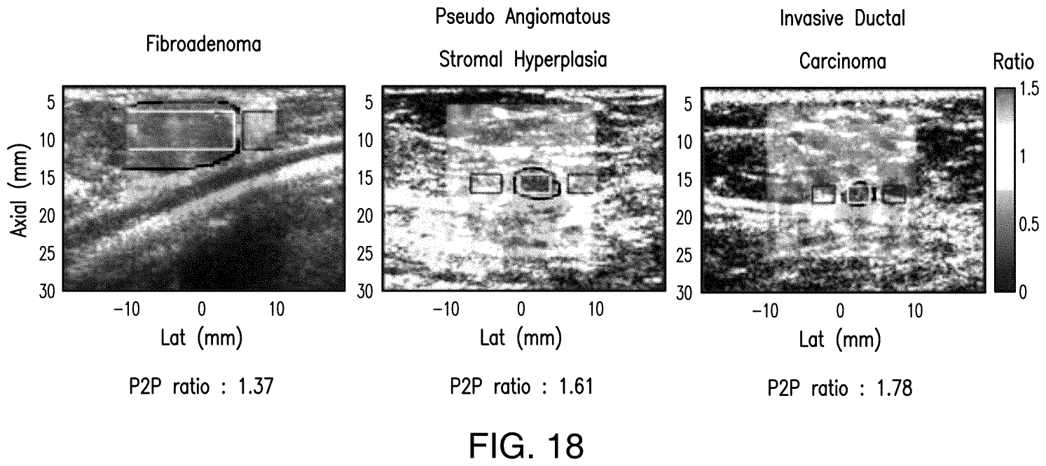

FIG. 16 shows normalized P2PD images of a representative mouse tumor at 1, 2, 3, and 4 weeks post-injection of tumor cell. P2PD images were overlaid on the B-mode ultrasound image and were normalized by generating depth-dependent profiles from the leftmost 2 mm lateral FOV in the health tissue (i.e., background). Two observations are notable. First, the tumor grew over time. Second, P2P displacements at the tumor with respect to the healthy tissue were getting lower over time. These observations were quantitatively demonstrated in FIG. 17, which shows that tumor diameters and P2PD ratios of healthy tissue over tumor increased over time. The median P2PD was 3.0, 5.1, 6.1, and 7.7 at 1, 2, 3, and 4 weeks, respectively. P2PD ratio was calculated using the ROI shown in FIG. 16.

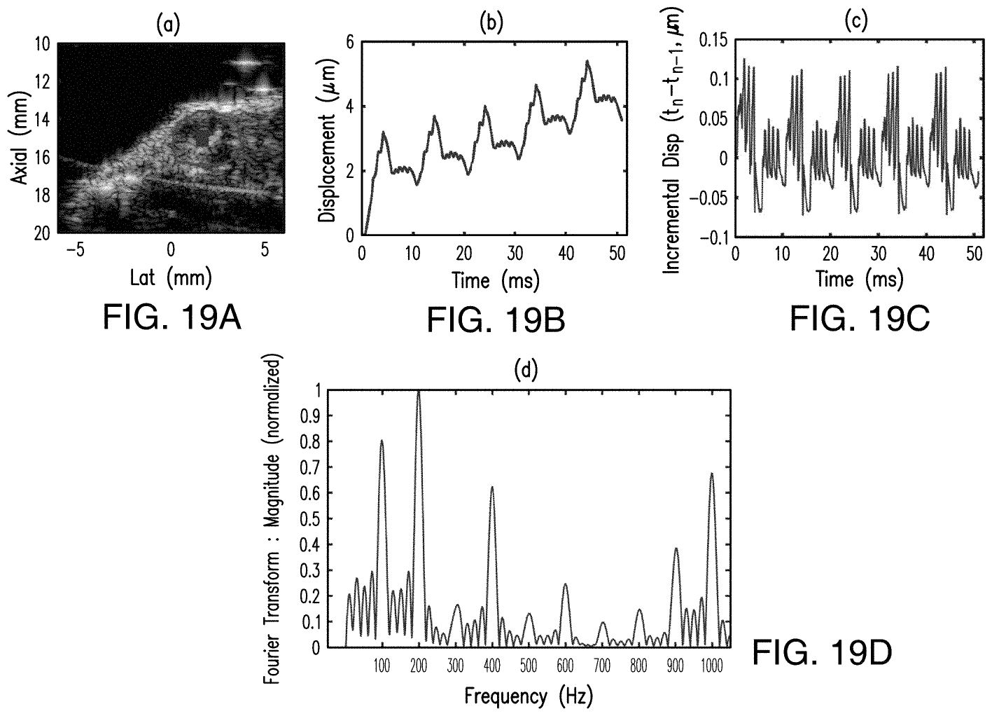

FIG. 19 shows normalized P2PD image overlaid on the B-mode ultrasound image of 23 years old Fibroadenoma (FA), 65 years old pseudo angiomatous stromal hyperplasia (PASH), and 54 years old invasive ductal carcinoma (IDC) patients. The median P2PD ratio of healthy tissue over tumor was 1.37, 1.61, and 1.78 in patients with FA, PASH, and IDC, respectively. ST-HMI was able to detect as small as 4 mm tumors (IDC patients).

A novel ARF-based method, named ST-HMI, to assess the mechanical properties of tissue has been presented. This novel method uses a single transducer to generate and track harmonic oscillation and is different from the HSWI method [54]. ST-HMI assesses mechanical properties at the ARF-ROE instead of shear wave propagation as it is done in HSWI. ST-HMI can provide better mechanical resolution and able to image deeper organs and/or stiffer tissue compared to HSWI. Another potential benefit of ST-HMI compared to the pulsed ARF-based method is that input oscillation can easily be filtered out from different motion artifacts. Systematically evaluating the robustness of ST-HMI against noise and comparing ST-HMI to shear wave-based methods in terms of mechanical resolution, performance in heterogeneous media, and the maximum focal depth are achieved by the disclosed subject matter.

The disclosed subject matter shows the feasibility of ST-HMI by experimenting in the commercially available phantoms, breast cancer mouse models, and patients with breast masses. ST-HMI derived P2PD images contrasted of 8, 10, 14, 15, 20, 40, and 60 kPa inclusions embedded in 5 kPa background (FIG. 13). Though 10±1 mm diameter was targeted to image all inclusions, the perceived size varied between inclusions. This can be due to the difference in transducer pressure on the surface of the phantoms during imaging, use of the same oscillation frequency and/or difference in true sizes. However, the perceived size was within the error range provided by the manufacturer.

The P2PD ratio of background over inclusion increased with increasing Young's moduli ratio with R² value of 0.93 (FIG. 14B). Linear regression was used to derive the R². The relationship between ST-HMI derived P2PD ratio, and Young's moduli ratio can not be linear. In a purely elastic material with a point source, the relationship is expected to be linear. However, complex inertia due to 3-D volumetric ARF and the presence of viscosity can make the relationship non-linear. The manufactured provided nominal median Young's modulus was used to calculate R² value. R² value can increase if the correct relationship and Young's modulus are used. In addition to high correlation, ST-HMI-derived contrast and CNR were statistically different between all pairs of inclusions, which suggests that ST-HMI can distinguish two inclusions when the minimum stiffness difference was 16.6% (12 versus 15 kPa). However, this minimum distinguishability was based on the nominal Young's modulus provided by the manufacturer. The ST-HMI detectability of inclusion can be improved by selecting an optimal frequency as the contrast, and CNR of ST-HMI-derived images depends on the oscillation frequency (FIGS. 15 and 16).

Other parameters like excitation pulse duty cycle, cycle number, and excitation pulse offset did not have a larger impact on CNR and contrast as the oscillation frequency. Then median percent change in contrast and CNR was less than 1% when duty cycle, cycle number, and excitation pulse offset was greater than 6.36%, 6, and 0 ms, respectively. These results are meaningful as it suggests that it is possible to perform ST-HMI with low exposure of ARF to the patients without compromising its performance. It can also help to implement ST-HMI in a low-cost ultrasound system which cannot generate a large number of excitation pulse due to memory and/or power supply constraints.

These results in the phantom were very encouraging. However, phantoms' environment was very idealistic. In vivo performance of ST-HMI was evaluated by monitoring longitudinal changes in stiffness of mouse breast cancer and patients with breast masses. In mice, tumors were getting stiffer with respect to the nearest healthy tissues as it grew in size. This in vivo assessment uses a high-frequency ultrasound probe for both generating ARF and tracking ARF-induced motion. P2PD ratio was not able to statistically distinguish between 2^{nd} a versus 3^{rd} week and 3^{rd} versus 4^{th} week. It can be due to the small number of mice (N=4). As lateral FOV of ST-HMI image using L22-14vXLF was 4 mm, acquisitions at different locations were stitched together to form the final image. It can introduce some errors. As the normalizing profile was generated from the homogeneous healthy tissue, the normalization process can induce error if there is no healthy tissue (axial depth of around 8-11 mm). To solve this problem, the normalizing profile was extrapolated by fitting it to a Gaussian function. It can still induce some errors. That's why ROI in the tumor was selected to match the available depth in the healthy tissue instead of the whole tumor. Finally, ST-HMI detected three different kinds of breast masses and showed that malignant breast mass (IDC) was stiffer than benign breast mass (FA and PASH) with respect to the nearest healthy tissue. However, more patients are needed to confirm this finding.

ST-HMI demonstrated very promising results in phantoms, mouse breast cancer models, and breast cancer patients.

The disclosed subject matter provides techniques for generating and tracking harmonic motion using a linear array transducer. ST-HMI contrasted six inclusions with varying stiffness using two commercially available phantoms. In the preclinical mouse test, the P2P ratio of the tumor over healthy tissues increased over time, suggesting that the tumor was getting stiffer with respect to the nearest healthy tissue. ST-HMI detected three different kinds of breast masses and showed that malignant breast mass (IDC) was stiffer than benign breast mass (FA and PASH) with respect to the nearest healthy tissue. These results suggest that ST-HMI assesses the mechanical properties of tissue via harmonic motion generation and tracking in the region of ARF excitation without observing shear wave propagation.

### Example 3: Monitoring Progression of Primary Tumor and Liver Metastases from Breast Cancer in a Mouse Model Using Single Transducer-Harmonic Motion Imaging

The disclosed subject matter provides techniques for monitoring the progression of both primary tumors and metastases in the liver using ST-HMI-derived displacements at multiple frequencies. Breast cancer is the most frequently diagnosed cancer, and 50% of all breast cancer patients can develop metastatic disease. Out of all metastatic breast cancer patients, liver metastases develop in approximately 50% of them. If left untreated, liver metastases are associated with poor survival ranging from 4 to 8 months. Early diagnosis or therapy monitoring can help to tailor treatment for individual patients. Change in liver metastases can be monitored via measuring the mechanical properties of the liver. To interrogate the mechanical properties at the “on-axis” to acoustic radiation force, single transducer-harmonic motion imaging (ST-HMI) transmits tracking pulses in-between the discrete excitation pulses. The disclosed techniques can provide systems and methods for monitoring liver metastases from breast cancer using ST-HMI-induced displacement at multiple frequencies in vivo.

The orthotropic, 4T1 breast cancer mouse model (N=6) was generated by injecting 1×105 4T1 breast cancer cells in the 4th inguinal mammary fat pad. ST-HMI of both primary cancer and liver was performed at a 7, 10, 13, 20, 26, and 32 days post-injection of tumor cells using Verasonics research system (Vantage 256, Verasonics Inc., Kirkland, Wash., USA) with L22-14vXLF (Vermon, Tours, France). ST-HMI was implemented with 13 discrete excitation pulses per period after sampling a continuous excitation pulse composed of the sum of sinusoids with 100:100:1000 Hz to interleave tracking pulses with a PRF of 15 kHz. The center frequency of the excitation and tracking pulse was 15.63 and 20.8 MHz, respectively. The displacements with respect to the reference frame were estimated using 1-D normalized cross-correlation with a kernel length of 0.77 mm. For each pixel, estimated displacements were filtered out using the 2nd order Butterworth filter followed by the generation of a peak-to-peak displacement (P2PD) image at 100:100:1000 Hz obtained by averaging P2PD values over 5 cycles. Median P2PD values over the region of interest in the primary tumor and liver were computed.

In primary tumors, P2PDs were decreased over time which indicates stiffening, and P2PD across time points were statistically different for 100-500 Hz (p<0.05, Kruskal-Wallis). P2PD at 100-500 Hz detected changes in tumor stiffness as early as between 7 versus 10 days. In the liver, P2PD was also decreased over time, but the rate was slower than the primary tumor, which was expected. The Kruskal-Wallis test indicates that the P2PD was different across time-points for 100-200 and 400-700 Hz and P2PD at 400 Hz detected changes in tumor stiffness as early as between 7 versus 10 days. A good correlation was found between the change in P2PD in the primary tumor versus the liver metastases, with the highest R² of 0.72 observed at 100 Hz.

FIGS. 19A-19D shows (FIG. 19A) Bmode ultrasound images of a mouse tumor, (FIG. 19B) example ST-HMI with multi-frequency excitation pulse induced displacement profile of a pixel in the tumor, (FIG. 19C) incremental or differential displacement profile, and (FIG. 19D) Fourier transform of the incremental displacement profile with peaks at 100:100:1000 Hz.

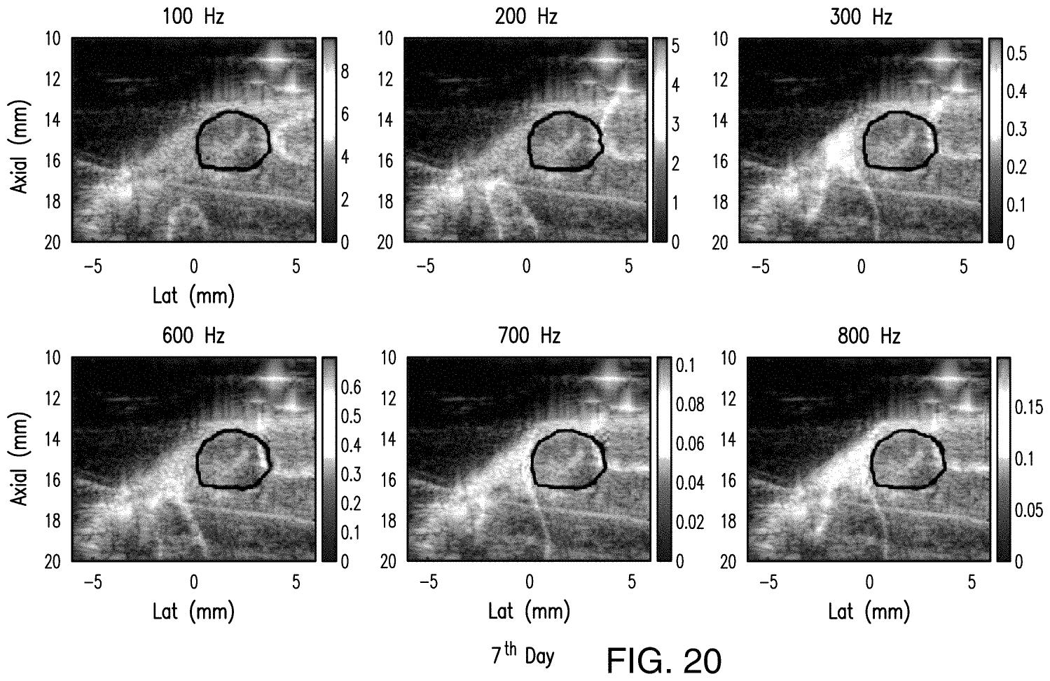

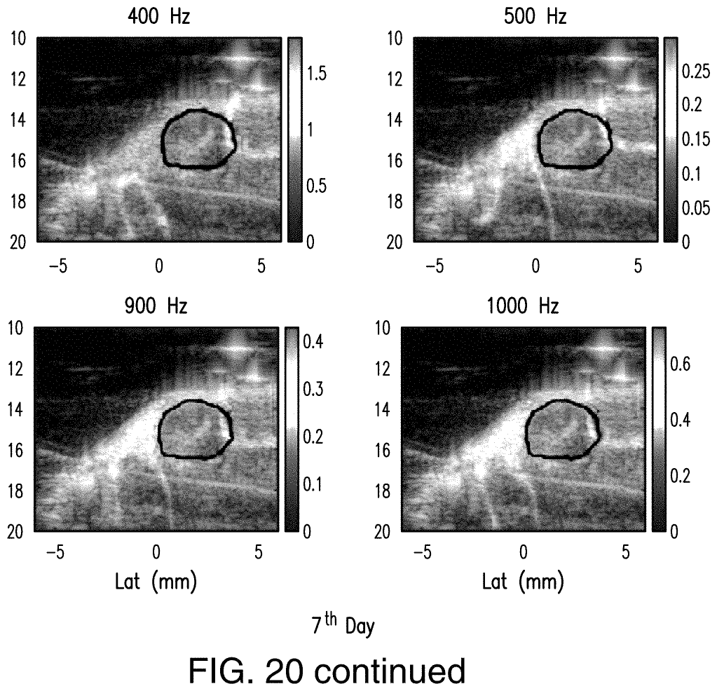

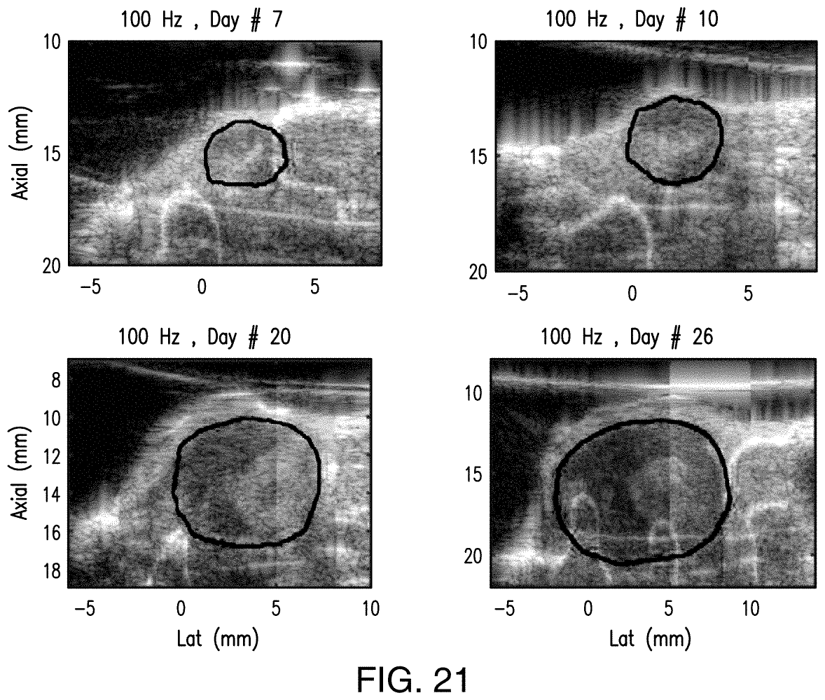

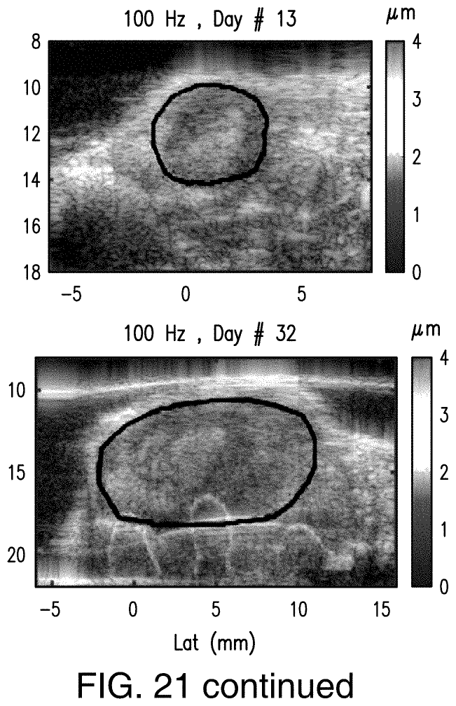

FIG. 20 shows example P2PD images at 100:100:1000 Hz overlaid on Bmode image of a mouse tumor at 7^{th} day post-injection of the tumor cells. FIG. 21 shows example P2PD images at 100 Hz overlaid on the Bmode image of a mouse tumor at 7, 10, 13, 20, 26, and 32^{nd} day post-injection of the tumor cells.

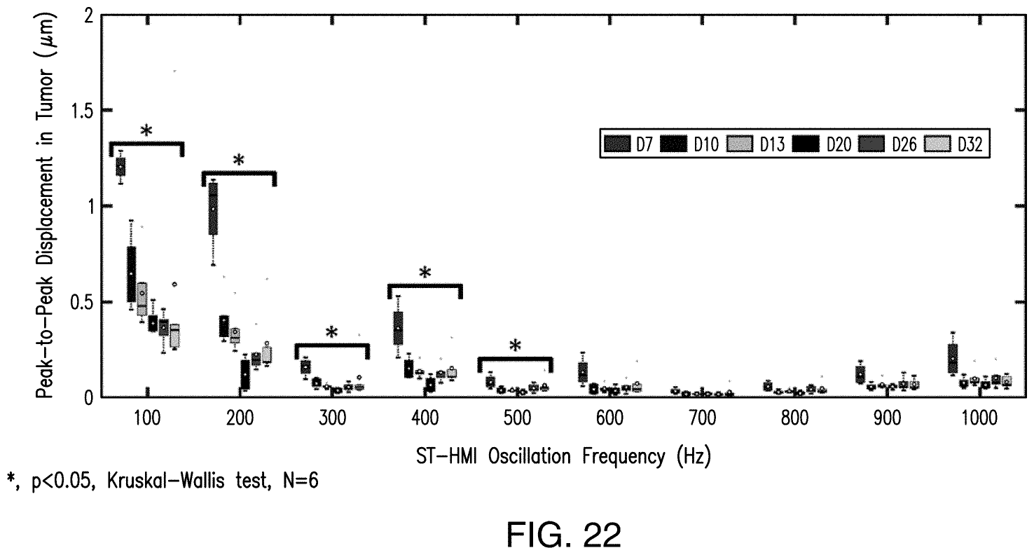

FIG. 22 shows a box plot of P2PD in tumor versus oscillation frequency with days (D7 to D32) after post-injection of tumor cells in the coded P2PD was statistically significant at 100-500 (shown by an asterisk), suggesting a change in P2PD, i.e., tumor stiffness over time.

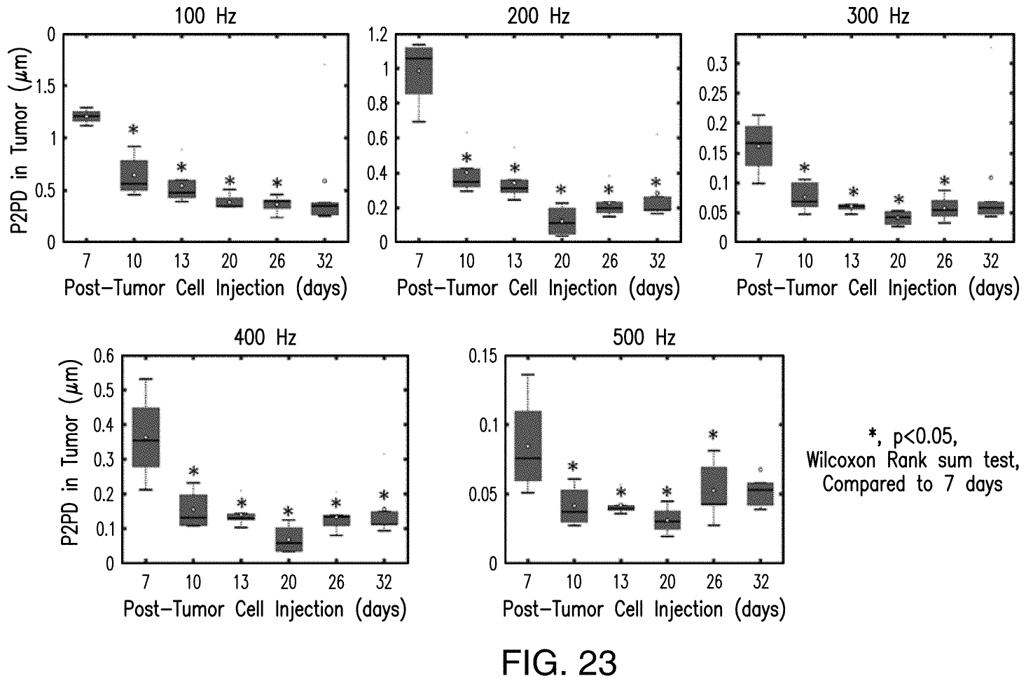

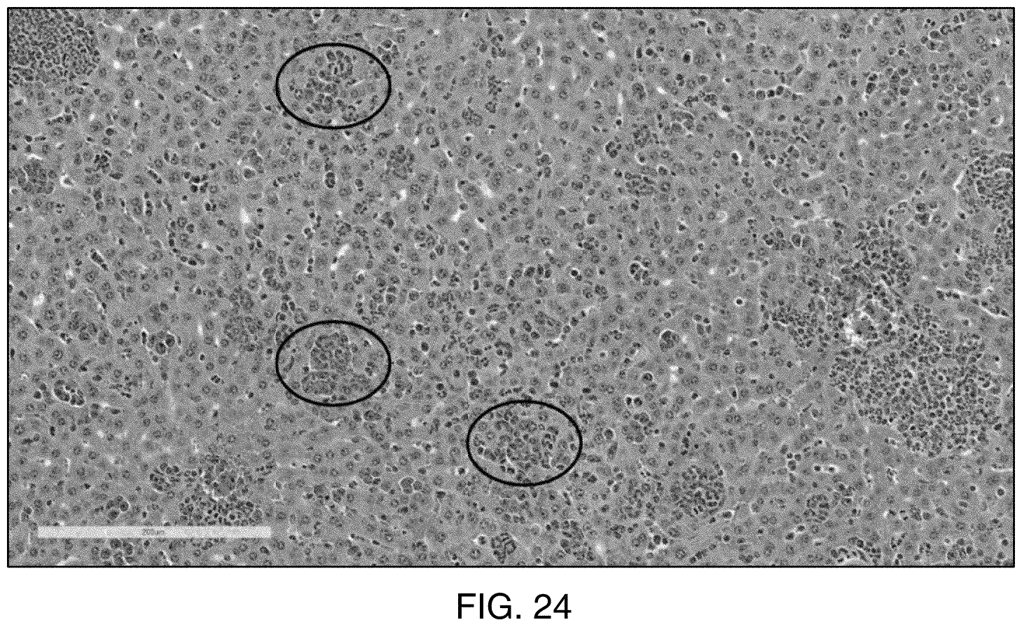

FIG. 23 provides a graph showing the statistical comparison of P2PD at 10, 13, 20, 26, and 32^{nd} day with P2PD at 7^{th} day post-injection of tumor cell. A statistically significant reduction in displacement is denoted by an asterisk. P2PD decreased over time suggests increasing tumor stiffness over time. ST-HMI was able to detect changes in tumor stiffness as early as between 7^{th} versus 10^{th} day.

FIG. 24 provides a histology image showing the presence of cancer cells in the liver, indicating diffuse metastases.

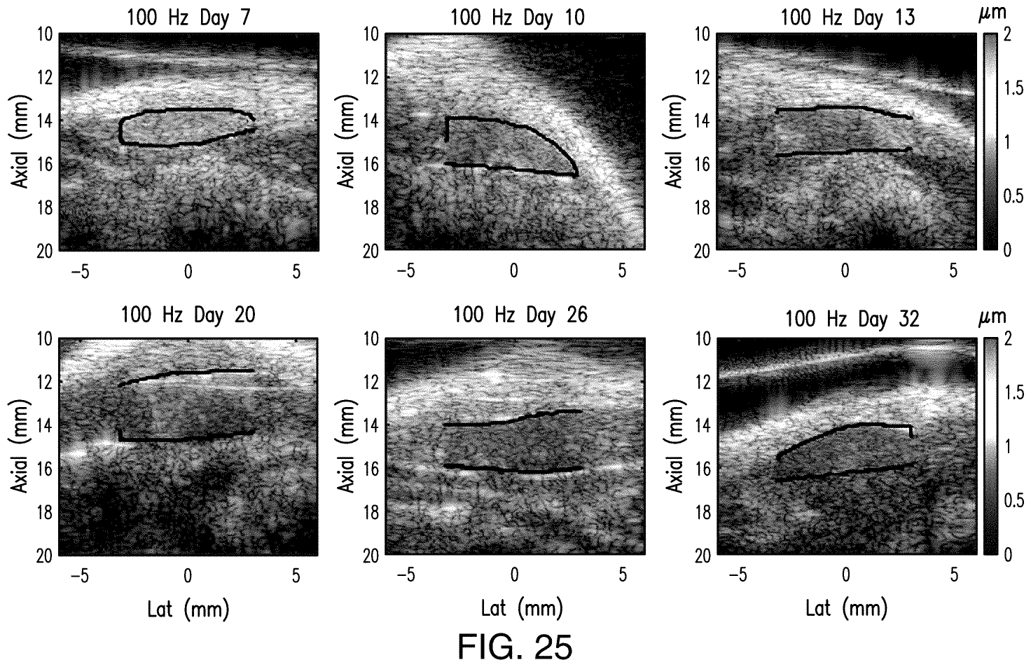

FIG. 25 provides images showing provides images showing example P2PD images at 100 Hz overlaid on Bmode image of a mouse liver at 7, 10, 13, 20, 26, and 32^{nd} day post-injection of the tumor cells. P2PD decreased over time.

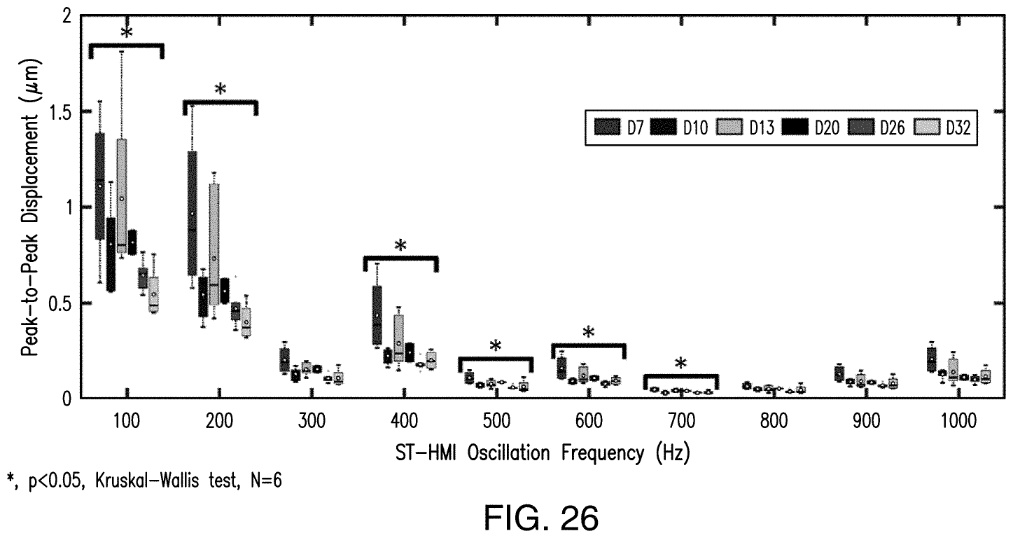

FIG. 26 provides a graph showing a box plot of P2PD in liver versus oscillation frequency with days (D7 . . . D32) after post-injection of tumor cells. P2PD was statistically significant at 100-200 and 400-700 Hz (shown by an asterisk), suggesting a change in P2PD, i.e., liver stiffness over time.

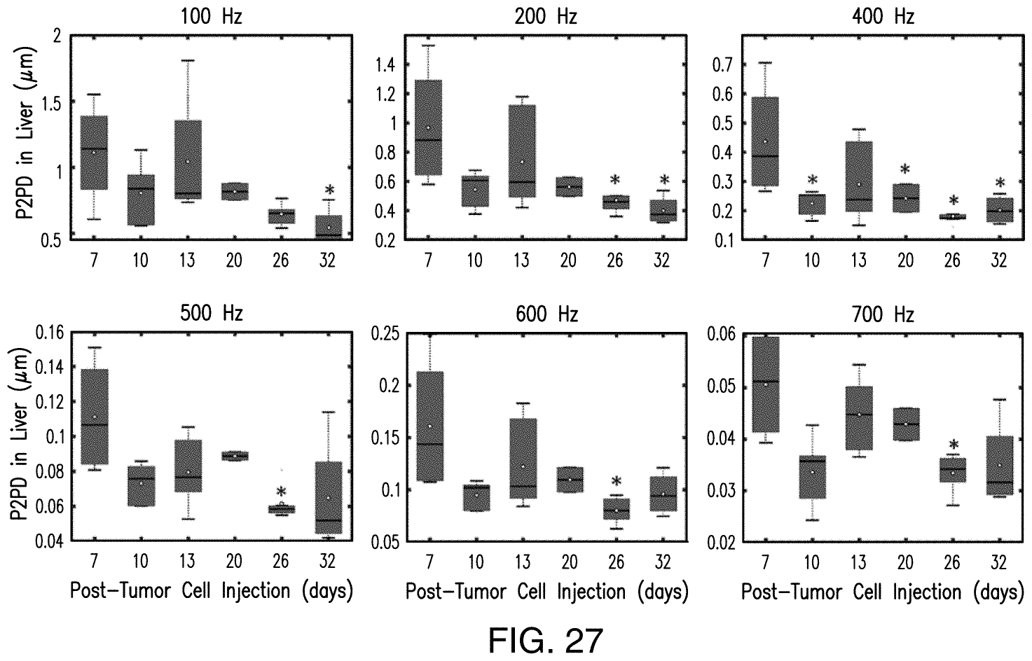

FIG. 27 provides graphs showing provides a graph showing the statistical comparison of P2PD of the liver at 10, 13, 20, 26, and 32^{nd} day with P2PD of the liver at 7^{th} day post-injection of tumor cell. A statistically significant reduction in displacement is denoted by an asterisk. P2PD decreased over time, suggesting increase liver stiffness over time. ST-HMI was able to detect changes in liver stiffness as early as between 7^{th} versus 10^{th} day (see 400 Hz).

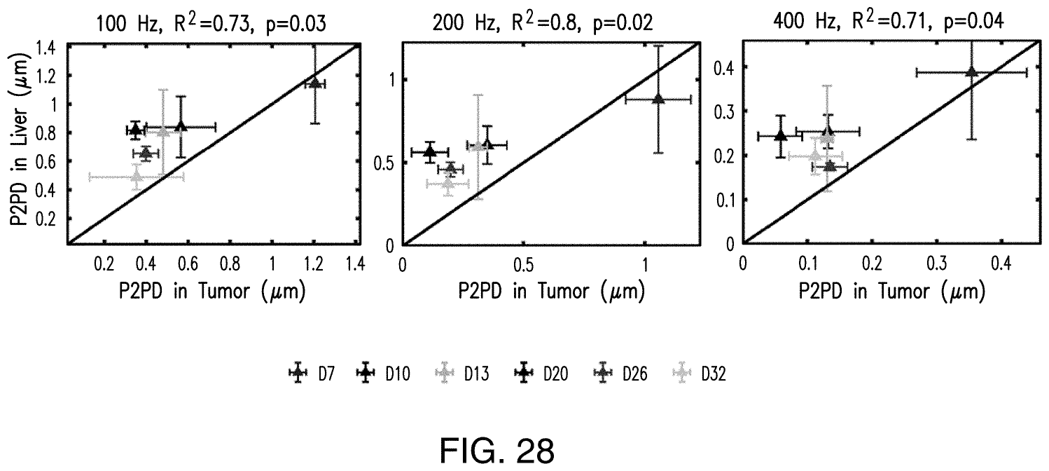

FIG. 28 shows linear regression between P2PD displacement at 100, 200, and 400 Hz between tumor versus liver. R² was greater than 0.70, suggesting a good correlation between liver metastases progression with primary tumor progression.

### Example 4: Generating Phase Velocity Map Corresponding to Frequencies of Excitation Pulses

The disclosed subject matter provides methods for generating a phase velocity map corresponding to the frequencies of the excitation pulse using the disclosed systems and techniques. Shear wave velocity as a function of frequency (i.e., phase velocity) can be used to phase velocity dispersion due to viscosity and ARF geometry. The selection of frequencies is important to correctly estimate mechanical properties and detect inclusion. Higher frequencies are better suited to reconstruct the shape of the stiffer inclusions and detect smaller inclusions with isotropic mechanical properties, and estimate fiber orientation and shear wave speed in anisotropic materials. Certain methods can generate pulsed ARF from a single ultrasound transducer or amplitude-modulated ARF (AMARF) from two ultrasound transducers to assess shear wave velocity as a function of frequency. Pulse ARF-generated shear waves with higher frequencies attenuate more and do not propagate further from the source. AMARF can be used to provide higher energy to a higher frequency, but the use of two transducers makes the system complex for diagnostic use. So, there is a need for generating AMARF and tracking shear waves using a single transducer to provide higher energy to a higher frequency and generate shear wave velocity images at higher frequencies.

A phase velocity map was generated based on the frequencies of the excitation pulse. Beamformed radiofrequency (RF) data was generated by performing delay-and-sum beamforming. Estimate displacements were induced by the AM-ARF based on the RF data. A two-dimensional interpolation was applied to the estimated displacements, and differential displacements between successive time points were calculated based on interpolated displacements. After applying directional filtering, one dimension Fourier transform along time dimension was applied to select frequencies of the excitation pulse, and two-dimensional Fourier transform along spatial dimension was applied at the selected frequency. Phase velocity at the selected frequency was calculated using a relationship between wavenumber and temporal frequency or linear regression of phase versus distance. Quantitative mechanical properties (e.g., viscosity and elasticity) were generated by fitting phase velocity versus frequency relationship derived analytically or empirically using rheological models.

Phase velocities at multiple frequencies were generated at two orthogonal directions by mechanically rotating a linear array transducer or electronically rotating point spread function using a 1.5D or matrix array. Phase velocities at the orthogonal directions were used to derive a degree of anisotropy as a function of frequency.

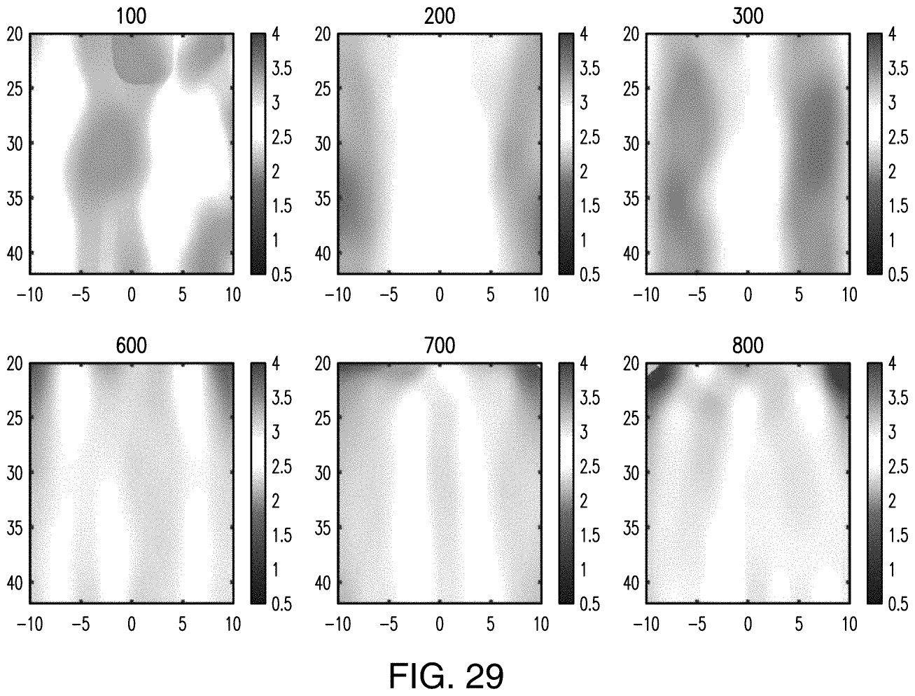

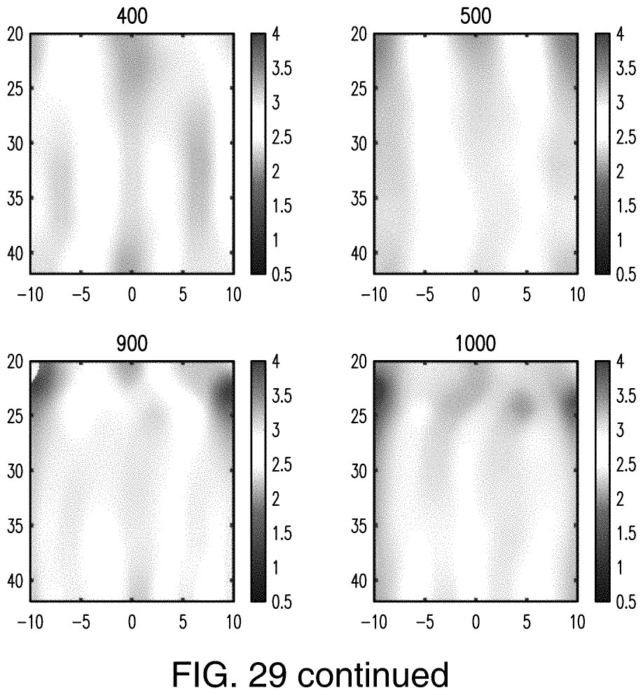

FIG. 29 provides images showing phase velocity (ms⁻¹) map at 100:100:1000 Hz of homogeneous background region of a commercially available phantom. The manufacture provided Young's modulus of the background is 18 kPa. The bar represents ms⁻¹.

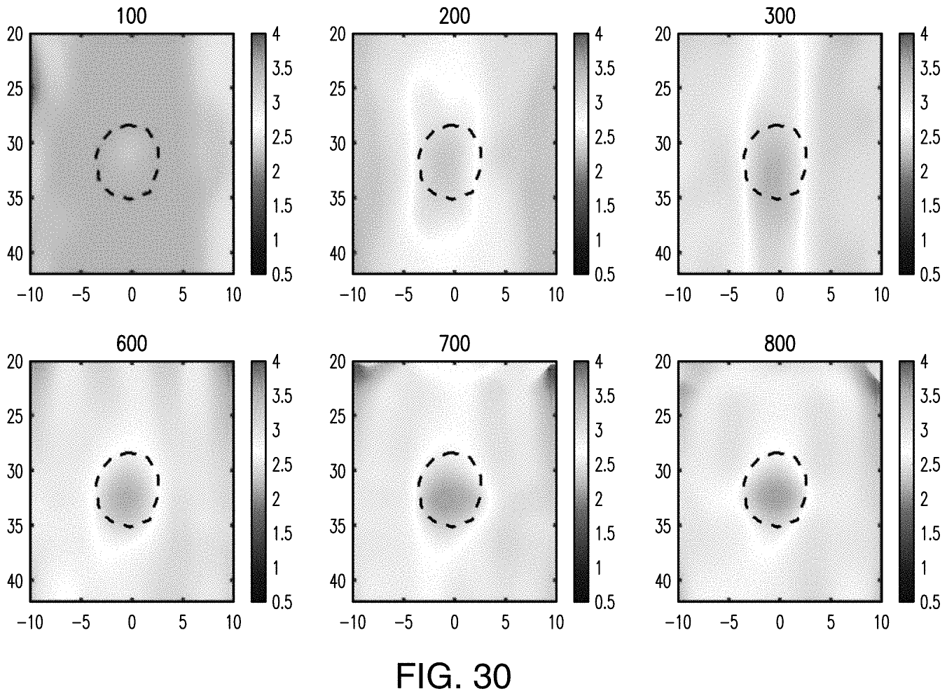

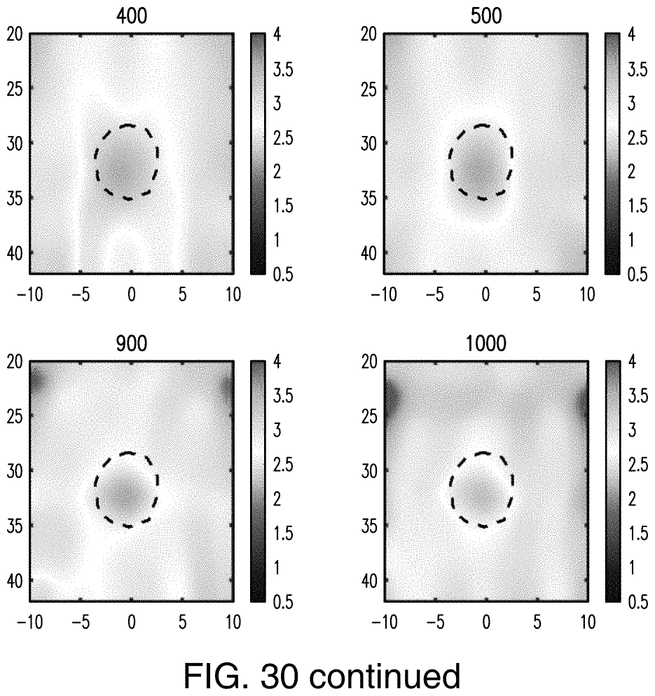

FIG. 30 provides images showing phase velocity (ms⁻¹) map at 100:100:1000 Hz of 6.5 mm 6 kPa inclusion embedded in 18 kPa background of a commercially available phantom. The stiffness (kPa) is given in terms of Young's modulus. The bar represents ms⁻¹. Black dashed contours represent inclusion boundaries derived from the Bmode ultrasound image.

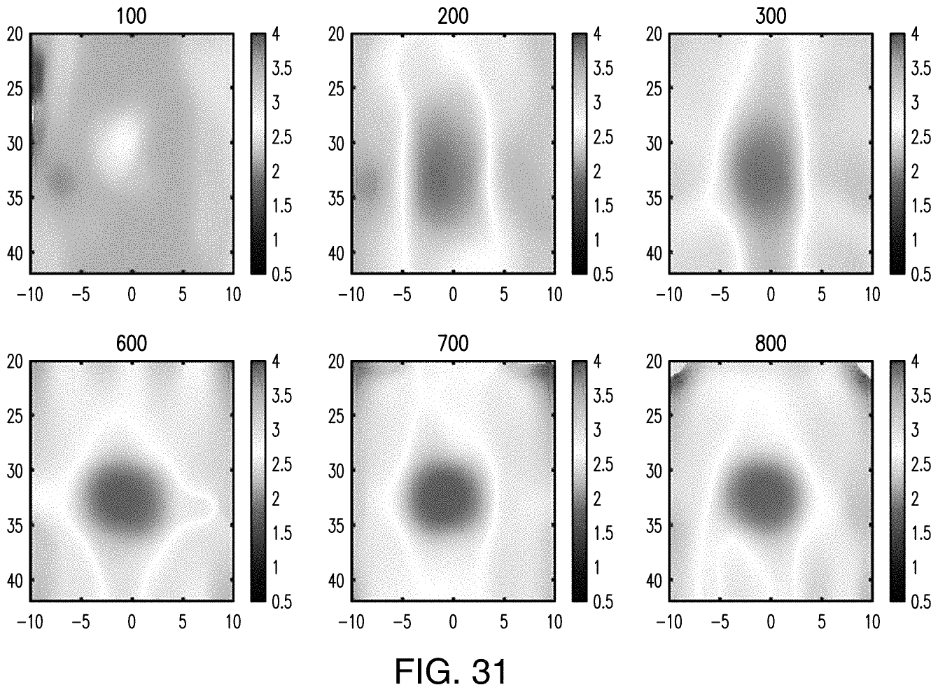

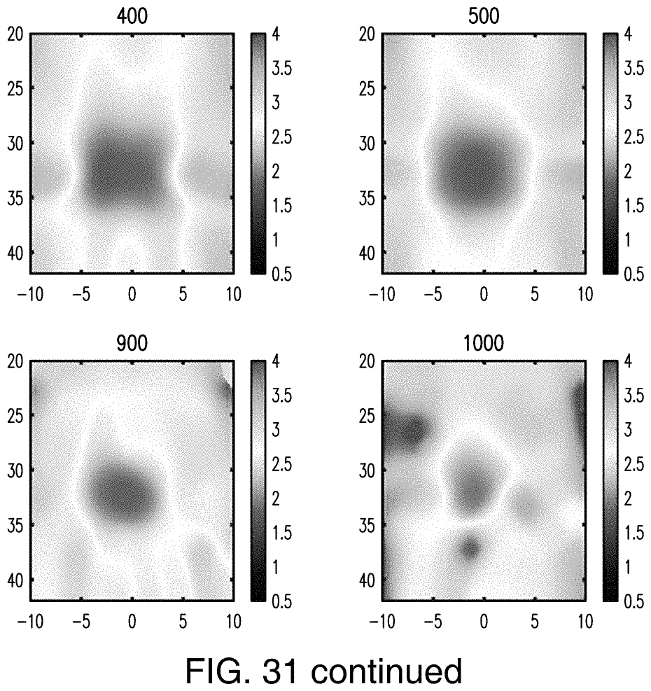

FIG. 31 provides images showing phase velocity (ms⁻¹) map at 100:100:1000 Hz of 10.4 mm 6 kPa inclusion embedded in 18 kPa background of a commercially available phantom. The stiffness (kPa) is given in terms of Young's modulus. The bar represents ms⁻¹.

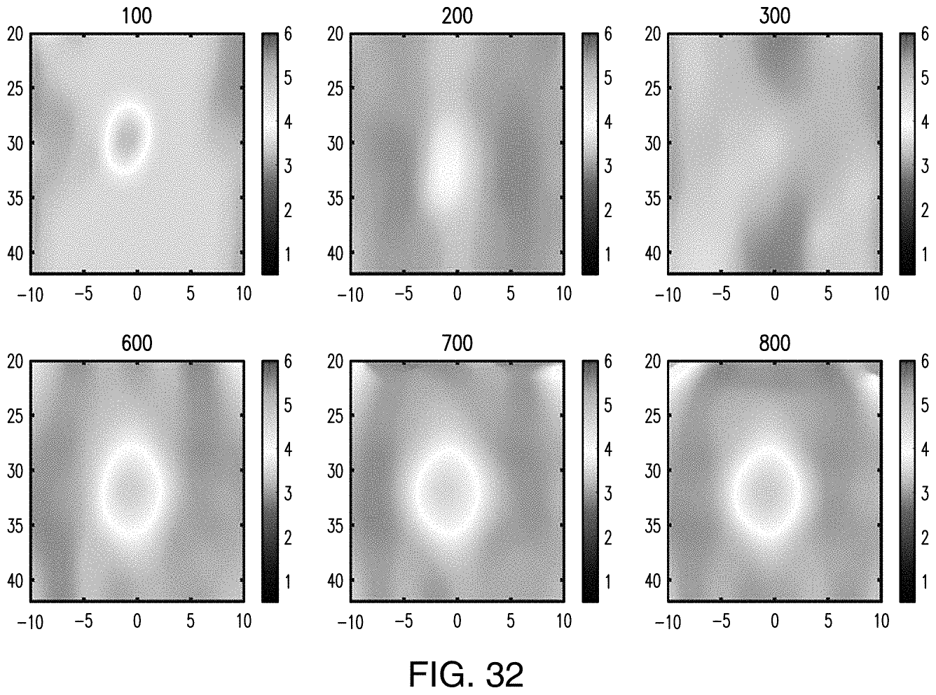

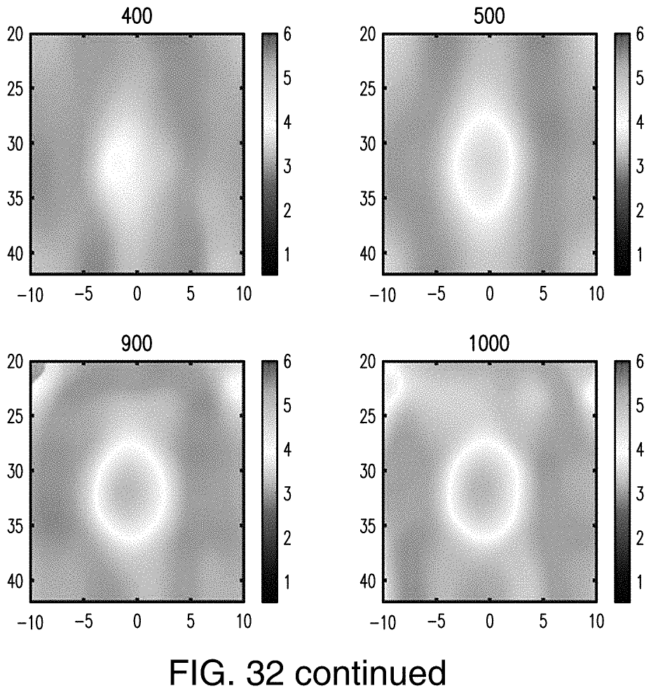

FIG. 32 provides images showing phase velocity (ms⁻¹) map at 100:100:1000 Hz of 6.5 mm 70 kPa inclusion embedded in 18 kPa background of a commercially available phantom. The stiffness (kPa) is given in terms of Young's modulus. The bar represents ms⁻¹.

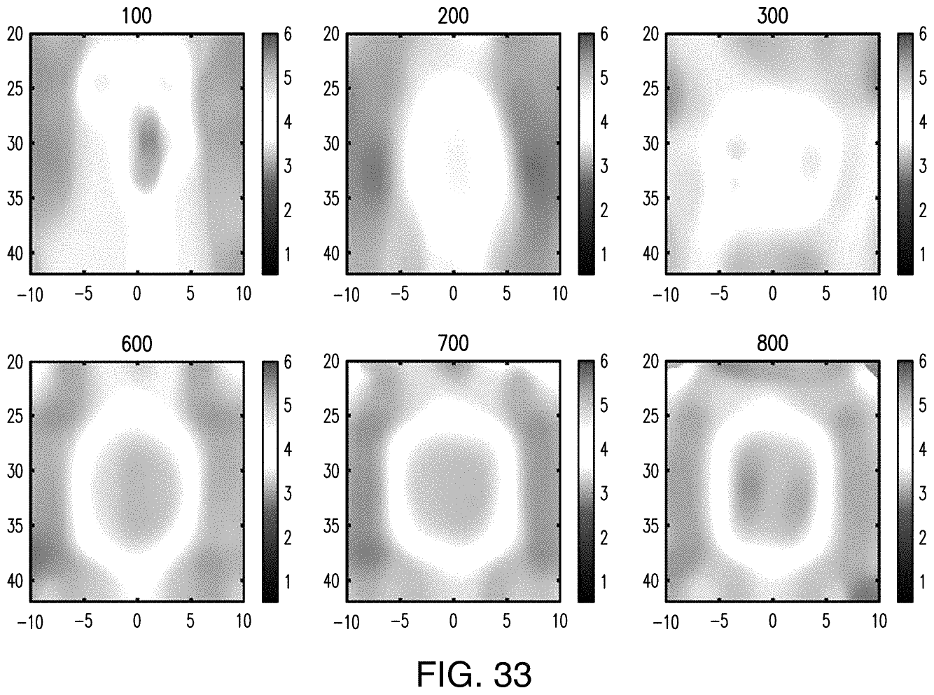

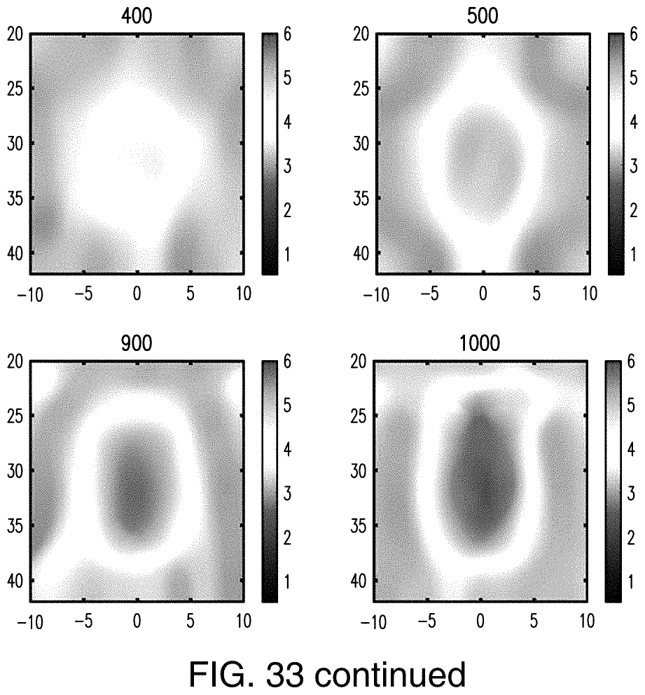

FIG. 33 shows a phase velocity (ms⁻¹) map at 100:100:1000 Hz of 10.4 mm 70 kPa inclusion embedded in 18 kPa background of a commercially available phantom. The stiffness (kPa) is given in terms of Young's modulus. The bar represents ms⁻¹.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A system for single transducer harmonic motion imaging, comprising:
a transducer configured to
generate an amplitude-modulated acoustic radiation force (AM-ARF) by sinusoidally modulating a duration of an excitation pulse of an acoustic radiation force, and
induce a harmonic motion on a target tissue using the AM-ARF, and simultaneously track the harmonic motion by collecting a tracking pulse, wherein the tracking pulse is interleaved between the excitation pulse.

2. The system of claim 1, the system further comprises a processor configured to estimate the mechanical properties of the target tissue based on the tacked harmonic motion.

3. The system of claim 1, wherein the excitation pulse includes a sum of sinusoids with about 100-2000 Hz frequencies.

4. The system of claim 3, wherein the processor is configured to generate a displacement map corresponding to the frequencies of the excitation pulse.

5. The system of claim 1, wherein the processor is configured to
generate beamformed radiofrequency (RF) data by performing delay-and-sum beamforming,
estimate displacements induced by the AM-ARF based on the RF data,
perform a two-dimensional interpolation on the estimated displacements,
calculate differential displacements between successive time points based on interpolated displacements,
filter the differential displacements for target frequencies, and
generate peak-to-peak displacement (P2PD) image based on the filtered differential displacements.

6. The system of claim 1, wherein the target tissue is selected from the group consisting of cancer, tumor, brain tissue, liver tissue, pancreatic tissue, breast tissue, prostate tissue, heart tissue, arterial tissue, renal tissue, and combinations thereof.

7. The system of claim 1, wherein a cycle of the excitation pulse is at least about 2 cycles.

8. The system of claim 1, wherein a cycle of the tracking pulse is about 2 cycles.

9. The system of claim 1, wherein a pulse repetition frequency (PRF) of the tracking pulse is from about 10 kHz to about 20 kHz.

10. The system of claim 1, wherein the mechanical properties include stiffness, Young's modulus, elasticity, viscosity, porosity, permeability, a degree of anisotropy, or combinations thereof.

11. The system of claim 1, wherein the transducer is configured to generate AM-ARF-induced displacements at a plurality of frequencies simultaneously.

12. The system of claim 1, wherein the transducer is configured to generate peak-to-peak displacement (P2PD) at a predetermined frequency at two orthogonal directions by mechanically rotating a linear array transducer or electronically rotating a point spread function using a matrix array or a transducer with more than one element in the elevational direction or a row-column array, wherein the point spread function defines a shape of an ultrasound beam.

13. The system of claim 12, a degree of anisotropy can be calculated based on the P2PD at the orthogonal directions.

14. The system of claim 1, a plurality of peak-to-peak displacements (P2PDs) at more than one frequencies can be generated at two orthogonal directions by mechanically rotating a linear array transducer or electronically rotating point spread function using a matrix array or a transducer with more than one element in the elevational direction or a row-column array, wherein a degree of anisotropy as a function of frequency is derived from the P2PDs at the orthogonal directions can be used to derive.

15. The system of claim 1, the processor is configured to calculate a degree of anisotropy by fitting a peak-to-peak displacement (P2PD) versus frequency relationship derived analytically or empirically using an anisotropic material model without rotating transducer or point spread function.

16. A method for single transducer harmonic motion imaging, comprising:
generating an amplitude-modulated acoustic radiation force (AM-ARF) by sinusoidally modulating a duration of an excitation pulse of an acoustic radiation force;
inducing a harmonic motion on a target tissue using the AM-ARF;
simultaneously tracking the harmonic motion by collecting a tracking pulse, wherein the tracking pulse is interleaved between the excitation pulse; and
estimating the mechanical properties of the target tissue based on the tracked harmonic motion.

17. The method of claim 16, further comprising
generating beamformed radiofrequency (RF) data by performing delay-and-sum beamforming;
estimating displacements induced by the AM-ARF based on the RF data;
performing a two-dimensional interpolation on the estimated displacements;
calculating differential displacements between successive time points based on interpolated displacements;
filtering the differential displacements for target frequencies; and
generating peak-to-peak displacement (P2PD) image based on the filtered differential displacements.

18. The method of claim 16, wherein the target tissue is selected from the group consisting of cancer, tumor, brain tissue, liver tissue, pancreatic tissue, breast tissue, prostate tissue, heart tissue, arterial tissue, renal tissue, and combinations thereof.

19. The method of claim 18, further comprising
determining a metastatic location and a metastatic level based on the estimated mechanical properties; and
administering a treatment to the metastatic location based on the metastatic level.

20. The method of claim 16, wherein the mechanical properties include stiffness, Young's modulus, elasticity, viscosity, porosity, permeability, a degree of anisotropy, or combinations thereof.

21. The method of claim 19, wherein the treatment includes anti-tumor treatment, anti-cancer treatment, chemotherapy, immunotherapy, radiation, surgery, or combinations thereof.

22. The method of claim 16, further comprising generating AM-ARF-induced displacements at a plurality of frequencies simultaneously.

23. The method of 16, further comprising generating a displacement map corresponding to the frequencies of the excitation pulse.

24. The method of 16, wherein the excitation pulse includes a sum of sinusoids with about 100-2000 Hz frequencies.

25. The method of claim 16, wherein a pulse repetition frequency (PRF) of the tracking pulse is from about 10 kHz to about 25 kHz.

26. The method of claim 16, further comprising generating a peak-to-peak displacement (P2PD) at a predetermined frequency at two orthogonal directions by mechanically rotating a linear array transducer or electronically rotating a point spread function using a matrix array or a transducer with more than one element in the elevational direction or a row-column array.

27. The method of claim 26, further comprising calculating a degree of anisotropy based on the P2PD at the orthogonal directions.

28. A method for generating a phase velocity map, comprising:
generating an amplitude-modulated acoustic radiation force (AM-ARF) by sinusoidally modulating a duration of an excitation pulse of an acoustic radiation force, wherein the excitation pulse includes at least about 2 cycles;
inducing shear waves at one or more frequencies simultaneously on a target tissue using the AM-ARF;
tracking the shear wave by collecting a tracking plane wave frames at a plurality angles with a frame rate between about 10 to about 20 kHz or collecting a focused pulse at a pulse repetition frequency of from about 10 to about 20 kHz between the excitation pulses;
generating a phase velocity map based on the tracked shear wave; and
estimating mechanical properties of the target tissue based on the phase velocity map.

29. The method of claim 28, further comprising
generating beamformed radiofrequency (RF) data by performing delay-and-sum beamforming;
estimating displacements induced by the AM-ARF based on the RF data;
performing a two-dimensional interpolation on the estimated displacements;
calculating differential displacements between successive time points based on interpolated displacements;
performing a filtering on the calculated differential displacements to remove reflected shear waves;
performing one dimensional Fourier transformation on the filtered differential displacements along time dimension to select a frequency of the excitation pulse;
performing two-dimensional Fourier transform along spatial dimension at the selected frequency of the excitation pulse; and
calculating a phase velocity at the selected frequency based on properties of the time and spatially Fourier transformed differential displacements, wherein the properties of the time and spatially Fourier transformed differential displacements include a wave number, a temporal frequency, a linear regression of phase versus distance or combinations thereof.

30. The method of claim 29, further comprising generating quantitative mechanical properties of the target tissue based on a P2PD versus frequency relationship or a phase velocity versus frequency relationship using a rheological model, wherein the rheological model is selected from the group consisting of a Maxwell model, a Kelvin-Voigt model, a standard linear solid model, a Burgers model, a generalized Maxwell model, and a Prony series.

31. The method of claim 28, wherein the shear waves at one or more frequencies are generated at two orthogonal directions by mechanically rotating a linear array transducer or electronically rotating point spread function using a matrix array or a transducer with more than one element in the elevational direction or a row-column array.

32. The method of claim 31, wherein the shear waves at the orthogonal directions are used to derive a degree of anisotropy as a function of frequency.

33. The method of claim 28, wherein the target tissue is selected from the group consisting of cancer, tumor, brain tissue, liver tissue, pancreatic tissue, breast tissue, prostate tissue, heart tissue, arterial tissue, renal tissue, and combinations thereof.

34. The method of claim 28, further comprising
determining a metastatic location and a metastatic level based on the estimated mechanical properties; and
administering a treatment to the metastatic location based on the metastatic level.

35. The method of claim 28, wherein the mechanical properties include stiffness, Young's modulus, elasticity, viscosity, porosity, permeability, a degree of anisotropy, or combinations thereof.

36. The method of claim 32, wherein the treatment includes anti-tumor treatment, anti-cancer treatment, chemotherapy, immunotherapy, radiation, surgery, or combinations thereof.
